SEQUENCE LISTING (89 sequences)

SEQ ID NO: 1 - DNA, 25 bp, Artificial Sequence
  Oligonucleotide
ttatcccgagaattcagacagtcac

SEQ ID NO: 2 - DNA, 25 bp, Artificial Sequence
  Oligonucleotide
aaccgctacaaggcggggcaccaca

SEQ ID NO: 3 - DNA, 40 bp, Artificial Sequence
  Oligonucleotide
gtgactgtctgaattttatcccgagaattcagacagtcac

SEQ ID NO: 4 - DNA, 40 bp, Artificial Sequence
  Oligonucleotide
tgtggtgccccgcctaaccgctacaaggcggggcaccaca

SEQ ID NO: 5 - DNA, 37 bp, Artificial Sequence
  Oligonucleotide
tgtgatcgacggttatcccgagaattcagacagtcac

SEQ ID NO: 6 - DNA, 36 bp, Artificial Sequence
  Oligonucleotide
atattcatgtgaaccgctacaaggcggggcaccaca

SEQ ID NO: 7 - DNA, 69 bp, Artificial Sequence
  Oligonucleotide
gtgactgtctgaattaatgatacggcgaccaccgagatctacacttatcccgagaattcagacagtcac

SEQ ID NO: 8 - DNA, 64 bp, Artificial Sequence
  Oligonucleotide
tgtggtgccccgcctcaagcagaagacggcatacgagataaccgctacaaggcggggcaccaca

SEQ ID NO: 9 - DNA, 20 bp, Artificial Sequence
  Oligonucleotide
aatgatacggcgaccaccga

SEQ ID NO: 10 - DNA, 20 bp, Artificial Sequence
  Oligonucleotide
caagcagaagacggcatacg

SEQ ID NO: 11 - DNA, 44 bp, Artificial Sequence
  Oligonucleotide
ttttgtgactgtctgttatcccgagaattcagacagtcacaaaa

SEQ ID NO: 12 - DNA, 44 bp, Artificial Sequence
  Oligonucleotide
gggtgtgactgtctgttatcccgagaattcagacagtcacaccc

SEQ ID NO: 13 - DNA, 44 bp, Artificial Sequence
  Oligonucleotide
ccctgtgactgtctgttatcccgagaattcagacagtcacaggg

SEQ ID NO: 14 - DNA, 44 bp, Artificial Sequence
  Oligonucleotide
aaatgtgactgtctgttatcccgagaattcagacagtcacattt

SEQ ID NO: 15 - DNA, 44 bp, Artificial Sequence
  Oligonucleotide
cgtggtgactgtctgttatcccgagaattcagacagtcaccacg

SEQ ID NO: 16 - DNA, 44 bp, Artificial Sequence
  Oligonucleotide
atgggtgactgtctgttatcccgagaattcagacagtcacccat

SEQ ID NO: 17 - DNA, 44 bp, Artificial Sequence
  Oligonucleotide
tacggtgactgtctgttatcccgagaattcagacagtcaccgta

SEQ ID NO: 18 - DNA, 44 bp, Artificial Sequence
  Oligonucleotide
gcaggtgactgtctgttatcccgagaattcagacagtcacctgc

SEQ ID NO: 19 - DNA, 44 bp, Artificial Sequence
  Oligonucleotide
actcgtgactgtctgttatcccgagaattcagacagtcacgagt

SEQ ID NO: 20 - DNA, 44 bp, Artificial Sequence
  Oligonucleotide
cagcgtgactgtctgttatcccgagaattcagacagtcacgctg

SEQ ID NO: 21 - DNA, 44 bp, Artificial Sequence
  Oligonucleotide
gtccgtgactgtctgttatcccgagaattcagacagtcacggac

SEQ ID NO: 22 - DNA, 44 bp, Artificial Sequence
  Oligonucleotide
tgacgtgactgtctgttatcccgagaattcagacagtcacgtca

SEQ ID NO: 23 - DNA, 44 bp, Artificial Sequence
  Oligonucleotide
gatagtgactgtctgttatcccgagaattcagacagtcactatc

SEQ ID NO: 24 - DNA, 44 bp, Artificial Sequence
  Oligonucleotide
tcgagtgactgtctgttatcccgagaattcagacagtcactcga

SEQ ID NO: 25 - DNA, 44 bp, Artificial Sequence
  Oligonucleotide
agcagtgactgtctgttatcccgagaattcagacagtcactgct

SEQ ID NO: 26 - DNA, 44 bp, Artificial Sequence
  Oligonucleotide
ctaagtgactgtctgttatcccgagaattcagacagtcacttag

SEQ ID NO: 27 - DNA, 43 bp, Artificial Sequence
  Oligonucleotide
agatcggannacactctttccctacacgacgctcttccgatct

SEQ ID NO: 28 - DNA, 43 bp, Artificial Sequence
  Oligonucleotide
agatcggannctcggcattcctgctgaaccgctcttccgatct

SEQ ID NO: 29 - DNA, 43 bp, Artificial Sequence
  Oligonucleotide
agatcnnnnnacactctttccctacacgacgctcttccgatct

SEQ ID NO: 30 - DNA, 43 bp, Artificial Sequence
  Oligonucleotide
agatcnnnnnctcggcattcctgctgaaccgctcttccgatct

SEQ ID NO: 31 - DNA, 45 bp, Artificial Sequence
  Oligonucleotide
agatcggnnnnnacactctttccctacacgacgctcttccgatct

SEQ ID NO: 32 - DNA, 45 bp, Artificial Sequence
  Oligonucleotide
agatcggnnnnnctcggcattcctgctgaaccgctcttccgatct

SEQ ID NO: 33 - DNA, 42 bp, Artificial Sequence
  Oligonucleotide
agatcggtnacactctttccctacacgacgctcttccgatct

SEQ ID NO: 34 - DNA, 42 bp, Artificial Sequence
  Oligonucleotide
agatcggcnctcggcattcctgctgaaccgctcttccgatct

SEQ ID NO: 35 - DNA, 47 bp, Artificial Sequence
  Oligonucleotide
gatcggaagagcgtacactctttccctacacgacgctcttccgatct

SEQ ID NO: 36 - DNA, 47 bp, Artificial Sequence
  Oligonucleotide
gatcggaagagcggctcggcattcctgctgaaccgctcttccgatct

SEQ ID NO: 37 - DNA, 48 bp, Artificial Sequence
  Oligonucleotide
tcgtgtagggaaagaacactctttccctacacgacgctcttccgatct

SEQ ID NO: 38 - DNA, 48 bp, Artificial Sequence
  Oligonucleotide
gttcagcaggaatgcctcggcattcctgctgaaccgctcttccgatct

SEQ ID NO: 39 - DNA, 14 bp, Artificial Sequence
  Oligonucleotide
agatcggaagagcg

SEQ ID NO: 40 - DNA, 48 bp, Artificial Sequence
  Oligonucleotide
agatcggaagagcgtacactctttccctacacgacgctcttccgatct

SEQ ID NO: 41 - DNA, 48 bp, Artificial Sequence
  Oligonucleotide
agatcggaagagcggctcggcattcctgctgaaccgctcttccgatct

SEQ ID NO: 42 - DNA, 56 bp, Artificial Sequence
  Oligonucleotide
aatgatacggcgaccaccgagatctacactctttccctacacgacgctcttccgat

SEQ ID NO: 43 - DNA, 58 bp, Artificial Sequence
  Oligonucleotide
caagcagaagacggcatacgagatcggtctcggcattcctgctgaaccgctcttccga

SEQ ID NO: 44 - DNA, 56 bp, Artificial Sequence
  Oligonucleotide
agcgtcgtgtagggaaagaacactctttccctacacgacgctcttccgatctaccc

SEQ ID NO: 45 - DNA, 14 bp, Artificial Sequence
  Oligonucleotide
gggtagatcggaag

SEQ ID NO: 46 - DNA, 56 bp, Artificial Sequence
  Oligonucleotide
agcgtcgtgtagggaaagaacactctttccctacacgacgctcttccgatctaggg

SEQ ID NO: 47 - DNA, 14 bp, Artificial Sequence
  Oligonucleotide
ccctagatcggaag

SEQ ID NO: 48 - DNA, 56 bp, Artificial Sequence
  Oligonucleotide
agcgtcgtgtagggaaagaacactctttccctacacgacgctcttccgatctccat

SEQ ID NO: 49 - DNA, 14 bp, Artificial Sequence
  Oligonucleotide
atggagatcggaag

SEQ ID NO: 50 - DNA, 56 bp, Artificial Sequence
  Oligonucleotide
agcgtcgtgtagggaaagaacactctttccctacacgacgctcttccgatctcgta

SEQ ID NO: 51 - DNA, 14 bp, Artificial Sequence
  Oligonucleotide
tacgagatcggaag

SEQ ID NO: 52 - DNA, 56 bp, Artificial Sequence
  Oligonucleotide
agcgtcgtgtagggaaagaacactctttccctacacgacgctcttccgatctgagt

SEQ ID NO: 53 - DNA, 14 bp, Artificial Sequence
  Oligonucleotide
actcagatcggaag

SEQ ID NO: 54 - DNA, 56 bp, Artificial Sequence
  Oligonucleotide
agcgtcgtgtagggaaagaacactctttccctacacgacgctcttccgatctgtca

SEQ ID NO: 55 - DNA, 14 bp, Artificial Sequence
  Oligonucleotide
tgacagatcggaag

SEQ ID NO: 56 - DNA, 56 bp, Artificial Sequence
  Oligonucleotide
agcgtcgtgtagggaaagaacactctttccctacacgacgctcttccgatcttatc

SEQ ID NO: 57 - DNA, 14 bp, Artificial Sequence
  Oligonucleotide
gataagatcggaag

SEQ ID NO: 58 - DNA, 56 bp, Artificial Sequence
  Oligonucleotide
agcgtcgtgtagggaaagaacactctttccctacacgacgctcttccgatctttag

SEQ ID NO: 59 - DNA, 14 bp, Artificial Sequence
  Oligonucleotide
ctaaagatcggaag

SEQ ID NO: 60 - DNA, 56 bp, Artificial Sequence
  Oligonucleotide
agcgtcgtgtagggaaagaacactctttccctacacgacgctcttccgatctaaaa

SEQ ID NO: 61 - DNA, 14 bp, Artificial Sequence
  Oligonucleotide
ttttagatcggaag

SEQ ID NO: 62 - DNA, 56 bp, Artificial Sequence
  Oligonucleotide
agcgtcgtgtagggaaagaacactctttccctacacgacgctcttccgatctattt

SEQ ID NO: 63 - DNA, 14 bp, Artificial Sequence
  Oligonucleotide
aaatagatcggaag

SEQ ID NO: 64 - DNA, 56 bp, Artificial Sequence
  Oligonucleotide
agcgtcgtgtagggaaagaacactctttccctacacgacgctcttccgatctcacg

SEQ ID NO: 65 - DNA, 14 bp, Artificial Sequence
  Oligonucleotide
cgtgagatcggaag

SEQ ID NO: 66 - DNA, 56 bp, Artificial Sequence
  Oligonucleotide
agcgtcgtgtagggaaagaacactctttccctacacgacgctcttccgatctctgc

SEQ ID NO: 67 - DNA, 14 bp, Artificial Sequence
  Oligonucleotide
gcagagatcggaag

SEQ ID NO: 68 - DNA, 56 bp, Artificial Sequence
  Oligonucleotide
agcgtcgtgtagggaaagaacactctttccctacacgacgctcttccgatctgctg

SEQ ID NO: 69 - DNA, 14 bp, Artificial Sequence
  Oligonucleotide
cagcagatcggaag

SEQ ID NO: 70 - DNA, 56 bp, Artificial Sequence
  Oligonucleotide
agcgtcgtgtagggaaagaacactctttccctacacgacgctcttccgatctggac

SEQ ID NO: 71 - DNA, 14 bp, Artificial Sequence
  Oligonucleotide
gtccagatcggaag

SEQ ID NO: 72 - DNA, 56 bp, Artificial Sequence
  Oligonucleotide
agcgtcgtgtagggaaagaacactctttccctacacgacgctcttccgatcttcga

SEQ ID NO: 73 - DNA, 14 bp, Artificial Sequence
  Oligonucleotide
tcgaagatcggaag

SEQ ID NO: 74 - DNA, 56 bp, Artificial Sequence
  Oligonucleotide
agcgtcgtgtagggaaagaacactctttccctacacgacgctcttccgatcttgct

SEQ ID NO: 75 - DNA, 14 bp, Artificial Sequence
  Oligonucleotide
agcaagatcggaag

SEQ ID NO: 76 - DNA, 48 bp, Artificial Sequence
  Oligonucleotide
ggaatagatgaagaccccgtcctagaacgaggtgtcttcatctattcc

SEQ ID NO: 77 - DNA, 48 bp, Artificial Sequence
  Oligonucleotide
gagtaccggatactttcagtttgaccttctttgaagtatccggtactc

SEQ ID NO: 78 - DNA, 48 bp, Artificial Sequence
  Oligonucleotide
ggaatannnnaagaccccgtcctagaacgaggtgtcttcatctattcc

SEQ ID NO: 79 - DNA, 48 bp, Artificial Sequence
  Oligonucleotide
gagtacnnnntactttcagtttgaccttctttgaagtatccggtactc

SEQ ID NO: 80 - DNA, 48 bp, Artificial Sequence
  Oligonucleotide
ggaatnnnnnnnnnncccgtcctagaacgaggtgtcttcatctattcc

SEQ ID NO: 81 - DNA, 48 bp, Artificial Sequence
  Oligonucleotide
gagtannnnnnnnnntcagtttgaccttctttgaagtatccggtactc

SEQ ID NO: 82 - DNA, 33 bp, Artificial Sequence
  Oligonucleotide
cccgtcctagaacgaggtgtcttcatctattcc

SEQ ID NO: 83 - DNA, 33 bp, Artificial Sequence
  Oligonucleotide
tcagtttgaccttctttgaagtatccggtactc

SEQ ID NO: 84 - DNA, 63 bp, Artificial Sequence
  Oligonucleotide
aatgatacggcgaccaccgagatctatgtgatcgacggttatcccgagaattcagacagtcac

SEQ ID NO: 85 - DNA, 60 bp, Artificial Sequence
  Oligonucleotide
caagcagaagacggcatacgagatatattcatgtgaaccgctacaaggcggggcaccaca

SEQ ID NO: 86 - DNA, 41 bp, Artificial Sequence
  Oligonucleotide
ccgtcgtgatcgacggttatcccgagaattcagacagtcac

SEQ ID NO: 87 - DNA, 25 bp, Artificial Sequence
  Oligonucleotide
gtgactgtctgaattctcgggataa

SEQ ID NO: 88 - DNA, 41 bp, Artificial Sequence
  Oligonucleotide
cacatgtattcatgtgaaccgctacaaggcggggcaccaca

SEQ ID NO: 89 - DNA, 25 bp, Artificial Sequence
  Oligonucleotide
tgtggtgccccgccttgtagcggtt

(19) United States
(12) Patent Application Publication
(10) Pub. No.: US 2011/0319290 A1
(43) Pub. Date: Dec. 29, 2011

(54) Methods and Compositions for Multiplex Sequencing

(75) Inventors: Christopher Raymond, Seattle, WA (US); Nurith Kurn, Palo Alto, CA (US); Jill Magnus, Seattle, WA (US)

(73) Assignee: NuGEN Technologies, Inc., San Carlos, CA (US)

(21) Appl. No.: 13/156,294

(22) Filed: Jun. 8, 2011

Related U.S. Application Data
(60) Provisional application No. 61/352,801, filed on Jun. 8, 2010.

(51) Int. Cl. C40B 40/06; C40B 30/04; C07H 21/02; C40B 30/00

(52) U.S. Cl. 506/9; 506/7; 506/16; 536/23.1

(57) ABSTRACT

Adapters are joined to target polynucleotides to create adapter-tagged polynucleotides. Adapter-tagged polynucleotides are sequenced simultaneously and sample sources are identified on the basis of barcode sequences.

### CROSS-REFERENCE

This application claims the benefit of U.S. Provisional Application No. 61/352,801, filed Jun. 8, 2010, which application is incorporated herein by reference.

### SEQUENCE LISTING

The instant application contains a Sequence Listing which has been submitted in ASCII format via EFS-Web and is hereby incorporated by reference in its entirety. Said ASCII copy, created on Jun. 8, 2011, is named 25115-741-201.txt and is 21 Kilobytes in size.

### BACKGROUND OF THE INVENTION

Large-scale sequence analysis of DNA can provide understanding of a wide range of biological phenomena related to states of health and disease, both in humans and in many economically important plants and animals, see e.g. Collins et al (2003), Nature, 422: 835-847; Service, Science, 311: 1544-1546 (2006); Hirschhorn et al (2005), Nature Reviews Genetics, 6: 95-108; National Cancer Institute, Report of Working Group on Biomedical Technology, “Recommendation for a Human Cancer Genome Project,” (February, 2005); Tringe et al (2005), Nature Reviews Genetics, 6: 805-814. The need for low-cost high-throughput sequencing and re-sequencing has led to the development of several new approaches that employ parallel analysis of many target DNA fragments simultaneously, e.g. Margulies et al, Nature, 437: 376-380 (2005); Shendure et al (2005), Science, 309: 1728-1732; Metzker (2005), Genome Research, 15: 1767-1776; Shendure et al (2004), Nature Reviews Genetics, 5: 335-344; Lapidus et al, U.S. patent publication US 2006/0024711; Drmanac et al, U.S. patent publication US 2005/0191656; Brenner et al, Nature Biotechnology, 18: 630-634 (2000); and the like. Such approaches reflect a variety of solutions for increasing target polynucleotide density and for obtaining increasing amounts of sequence information within each cycle of a particular sequence detection chemistry.

Given the complexity of the mixture of sequences in a given reactions, sequencing is typically restricted to one sample per reaction chamber. However, the number of bases read in a given reaction using these next generation sequencing technologies can be far greater than that actually needed to acquire the sequence information of interest, which essentially amounts to wasted sequencing space. Coupled with increasing desires to sequence samples from multiple sources, the expense of utilizing these technologies can quickly become prohibitive. Sequencing runs are also often limited in the number of separate reactions that can be run in parallel, which places further restrictions on the efficiency with which large numbers of samples can be processed.

Some approaches to resolve these challenges have involved the incorporation of additional identifier sequences into each of the target fragments analyzed. Where different sequences are used for different samples, sequencing of pooled samples can be followed by resolution of sequences into subsets corresponding to sample sources based on the added sequences. However, addition of sequences to resolve sample sources faces two challenges. Firstly, random errors in sequencing can make it impossible to correctly identify an appended identifier sequence with its sample source when such errors occur within appended sequences that are either too short or insufficiently dissimilar from sequences corresponding to other samples. Secondly, the addition of longer sequences to allow for such sequencing error takes up valuable sequencing space from target reads that can be as short as 20 bases. In view of these limitations, there is a need to increase the efficiency of next generation sequencing technologies such that samples can be sequenced in greater numbers, with greater identification accuracy, while maximizing the available sequencing space.

### SUMMARY OF THE INVENTION

In one aspect, the invention provides methods, compositions, and kits for multiplex sequencing. In one embodiment, the method comprises sequencing a plurality of target polynucleotides in a single reaction chamber, wherein said target polynucleotides are from two or more different samples; and identifying the sample from which each of said sequenced target polynucleotides is derived with an accuracy of at least 95% based on a single barcode contained in the sequence of said target polynucleotide. In some embodiments, the target polynucleotides comprise one or more sequences with which the sequencing reaction is calibrated. In some embodiments, each barcode differs from every other barcode at least three nucleotide positions. In some embodiments, the identification of sample source is accurate after the mutation or deletion of a nucleotide in the barcode.

In another aspect, the invention provides methods, compositions, and kits for producing adapter-tagged target polynucleotides from a plurality of independent samples. In one embodiment, the method comprises: (a) providing a plurality of first adapter oligonucleotides, wherein each of said first adapter oligonucleotides comprises at least one of a plurality of barcode sequences, wherein each barcode sequence of the plurality of barcode sequences differs from every other barcode sequence in said plurality of barcode sequences at least three nucleotide positions; and (b) joining at least one of said first adapter oligonucleotides to said target polynucleotides of each of said samples, such that no barcode sequence is joined to said target polynucleotides of more than one of said samples. In some embodiments, the method further comprises (c) joining at least one of a plurality of second adapter oligonucleotides to said target polynucleotides of each of said samples from step (b), such that at least some of said target polynucleotides comprise said first adapter oligonucleotide at one end and said second adapter oligonucleotide at the other end. One or more of the adapter oligonucleotides of the present invention can comprise SEQ ID NO: 1. One or more of the adapter oligonucleotides of the present invention can comprise SEQ ID NO: 2. One or more of the adapter oligonucleotides can comprise a hairpin structure. One or more of the adapter oligonucleotides can comprise an oligonucleotide duplex.

In some embodiments, the barcode sequences are at least 3 nucleotides in length. In some embodiments, the plurality of barcode sequences includes sequences selected from the group consisting of: AAA, TTT, CCC, and GGG. In some embodiments, the plurality of barcode sequences includes sequences selected from the group consisting of: AAAA, CTGC, GCTG, TGCT, ACCC, CGTA, GAGT, TTAG, AGGG, CCAT, GTCA, TATC, ATTT, CACG, GGAC, and TCGA. In some embodiments, the plurality of barcode sequences includes sequences selected from the group consisting of: AAAAA, AACCC, AAGGG, AATTT, ACACG, ACCAT, ACGTA, ACTGC, AGAGT, AGCTG, AGGAC, AGTCA, ATATC, ATCGA, ATGCT, ATTAG, CAACT, CACAG, CAGTC, CATGA, CCAAC, CCCCA, CCGGT, CCTTG, CGATA, CGCGC, CGGCG, CGTAT, CTAGG, CTCTT, CTGAA, CTTCC, GAAGC, GACTA, GAGAT, GATCG, GCATT, GCCGG, GCGCC, GCTAA, GGAAG, GGCCT, GGGGA, GGTTC, GTACA, GTCAC, GTGTG, GTTTT, TAATG, TACGT, TAGCA, TATAC, TCAGA, TCCTC, TCGAG, TCTCT, TGACC, TGCAA, TGGTT, TGTGG, TTAAT, TTCCG, TTGGC, and TTTTA.

In some embodiments, the method further comprises pooling the target polynucleotides from step (c). Target polynucleotides can be pooled based on the barcode sequences to which they are joined, such that all four bases are evenly represented at one or more positions along each barcode in the pool.

In some embodiments, target polynucleotides comprise fragmented sample polynucleotides. Fragmentation can comprise subjecting sample polynucleotides to acoustic sonication, and/or treating sample polynucleotides with one or more enzymes under conditions suitable for the one or more enzymes to generate random double-stranded nucleic acid breaks (which can include DNase I, Fragmentase, and variants thereof). In some embodiments, fragmentation comprises treating the sample polynucleotides with one or more restriction endonucleases. Fragments can have an average length of 10 to 10,000 nucleotides, such as an average length of 100-2,500 nucleotides, or 50-500 nucleotides. In some embodiments, samples comprise less than 500 ng of nucleic acid. Target polynucleotides can comprise genomic DNA, DNA produced by a primer extension reaction, cDNA, mitochondrial DNA, chloroplast DNA, plasmid DNA, bacterial artificial chromosomes, yeast artificial chromosomes, or a combination thereof.

In some embodiments, the method further comprises performing the step of extending one or more 3′ ends of the target polynucleotides, using the one or more joined adapter oligonucleotides as template. In some embodiments, the method further comprises amplifying the target polynucleotides after the extending step using a first primer and a second primer, wherein the first primer comprises a sequence that is hybridizable to at least a portion of the complement of one or more of the first adapter oligonucleotides, and further wherein the second primer comprises a sequence that is hybridizable to at least a portion of the complement of one or more of the second adapter oligonucleotides. One or more of the primers used in the amplification step can comprise SEQ ID NO: 1. One or more of the primers used in the amplification step can comprise SEQ ID NO: 2.

In some embodiments, each second adapter oligonucleotide comprises at least one of a plurality of barcode sequences, wherein each barcode sequence of the plurality of barcode sequences differs from every other barcode sequence in the plurality of barcode sequences at least three nucleotide positions. Pairs of first and second adapter oligonucleotides can comprise the same or different barcode sequences.

In some embodiments, the method further comprises sequencing one or more of the polynucleotides in a pool of target polynucleotides from independent samples. Sequencing can comprise extension of a sequencing primer comprising a sequence hybridizable to at least a portion of the complement of the first adapter oligonucleotide and/or the second adapter oligonucleotide. In some embodiments, the sequencing primer comprises SEQ ID NO: 1 or SEQ ID NO: 2. In some embodiments, sequencing comprises a calibration step, wherein calibration is based on each of the nucleotides at one or more nucleotide positions in the barcode sequences.

In some embodiments, the method further comprises identifying the sample from which a target polynucleotide is derived based on a barcode sequence to which it is joined.

In another aspect, the invention provides compositions for use in the described methods, comprising any one or more of the elements described herein. In one aspect, the invention provides a composition for multiplex sequencing. In one embodiment, the composition comprises a plurality of target polynucleotides, each target polynucleotide comprising one or more barcode sequences selected from a plurality of barcode sequences, wherein said target polynucleotides are from two or more different samples, and further wherein the sample from which each of said polynucleotides is derived can be identified in a combined sequencing reaction with an accuracy of at least 95% based on a single barcode contained in the sequence of said target polynucleotide.

In another aspect, the invention provides a composition useful in the generation of adapter-tagged target polynucleotides, comprising any one or more of the elements described herein. In one embodiment, the composition comprises a plurality of first adapter oligonucleotides, wherein each of said first adapter oligonucleotides comprises at least one of a plurality of barcode sequences, wherein each barcode sequence of the plurality of barcode sequences differs from every other barcode sequence in said plurality of barcode sequences at least three nucleotide positions. In some embodiments, the composition further comprises a plurality of second adapter oligonucleotides. In some embodiments, target polynucleotides are contained in a flow cell. First adapter oligonucleotides can be grouped in multiples of four such that all four bases are evenly represented at each position along each barcode. Where the second adapter oligonucleotide comprises a barcode, pairs of first and second adapter oligonucleotides can comprise the same or different barcode sequences. In some embodiments, the composition further comprises a first primer and a second primer, wherein said first primer comprises a sequence that is hybridizable to at least a portion of the complement of one or more of said first adapter oligonucleotides, and further wherein said second primers comprise a sequence that is hybridizable to at least a portion of the complement of one or more of said second adapter oligonucleotides. In some embodiments, the composition additionally comprises a sequencing primer comprising a sequence hybridizable to at least a portion of the complement of said first adapter oligonucleotide and/or said second adapter oligonucleotide.

In some embodiments, the composition comprises a plurality of first adapter oligonucleotides, wherein each of said first adapter oligonucleotides comprises a 5′ end comprising sequence A and a 3′ end comprising sequence A′, and further wherein A is hybridizable to A′, one of A or A′ comprises DNA, and the other of A or A′ comprises RNA and 5 or more terminal DNA nucleotides. In some embodiments, the composition further comprises a plurality of second adapter oligonucleotides, wherein each of said second adapter oligonucleotides comprises a 5′ end comprising sequence B and a 3′ end comprising sequence B′, and further wherein B is hybridizable to B′, one of B or B′ comprises DNA, and the other of B or B′ comprises RNA and 5 or more terminal DNA nucleotides.

In another aspect, the invention provides kits containing any one or more of the elements disclosed in the described methods and compositions. In one aspect, the invention provides a kit useful in the generation of adapter-tagged target polynucleotides. In one embodiment, the kit comprises a plurality of first adapter oligonucleotides, wherein each of said first adapter oligonucleotides comprises at least one of a plurality of barcode sequences, wherein each barcode sequence of the plurality of barcode sequences differs from every other barcode sequence in said plurality of barcode sequences at least three nucleotide positions, and instructions for using the same. In some embodiments, the kit further comprises a plurality of second adapter oligonucleotides. In some embodiments, the kit further comprises a first primer and a second primer, wherein said first primer comprises a sequence that is hybridizable to at least a portion of the complement of one or more of said first adapter oligonucleotides, and further wherein said second primers comprise a sequence that is hybridizable to at least a portion of the complement of one or more of said second adapter oligonucleotides. In some embodiments, the kit additionally comprises a sequencing primer comprising a sequence hybridizable to at least a portion of the complement of said first adapter oligonucleotide and/or said second adapter oligonucleotide. In some embodiments, the kit further comprises one or more of: (a) a DNA ligase, (b) a DNA-dependent DNA polymerase, (c) an RNA-dependent DNA polymerase, (d) random primers, (e) primers comprising at least 4 thymidines at the 3′ end, (f) a DNA endonuclease, (g) a DNA-dependent DNA polymerase having 3′ to 5′ exonuclease activity, (h) a plurality of primers, each primer having one of a plurality of selected sequences, (i) a DNA kinase, (j) a DNA exonuclease, (k) magnetic beads, (l) an enzyme comprising RNase H activity, (m) an RNA ligase, and (n) one or more buffers suitable for one or more of the elements contained in said kit.

In some embodiments, the kit comprises a plurality of first adapter oligonucleotides, wherein each of said first adapter oligonucleotides comprises a 5′ end comprising sequence A and a 3′ end comprising sequence A′, and further wherein A is hybridizable to A′, one of A or A′ comprises DNA, and the other of A or A′ comprises RNA and 5 or more terminal DNA nucleotides. In some embodiments, the kit further comprises a plurality of second adapter oligonucleotides, wherein each of said second adapter oligonucleotides comprises a 5′ end comprising sequence B and a 3′ end comprising sequence B′, and further wherein B is hybridizable to B′, one of B or B′ comprises DNA, and the other of B or B′ comprises RNA and 5 or more terminal DNA nucleotides.

In another aspect, the invention provides a method of producing adapter-tagged polynucleotides. In one embodiment, the method comprises: (a) providing a plurality of first adapter oligonucleotides, wherein each of said first adapter oligonucleotides comprises a 5′ end comprising sequence A and a 3′ end comprising sequence A′, and further wherein A is hybridizable to A′, one of A or A′ comprises DNA, and the other of A or A′ comprises RNA and 5 or more terminal DNA nucleotides; and, (b) joining at least one of said first adapter oligonucleotides to at least one of said target polynucleotides. Each of said first adapter oligonucleotides may comprise a barcode sequence. In some embodiments, the method further comprises the step of cleaving RNA with an enzyme that cleaves RNA from an RNA-DNA heteroduplex. In some embodiments, the method further comprises the step of extending one or more 3′ ends of said target polynucleotides, using said one or more joined adapter oligonucleotides as template. In some embodiments, the method comprises joining at least one of a plurality of second adapter oligonucleotides to said target polynucleotides of each of said samples from step (b), such that at least one of said target polynucleotides comprises said first adapter oligonucleotide at one end and said second adapter oligonucleotide at the other end. In some embodiments, each of said second adapter oligonucleotides comprises a 5′ end comprising sequence B and a 3′ end comprising sequence B′, and further wherein B is hybridizable to B′, one of B or B′ comprises DNA, and the other of B or B′ comprises RNA and 5 or more terminal DNA nucleotides. In some embodiments, each of said second adapter oligonucleotides comprises a barcode sequence.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a schematic drawing of one embodiment of the methods of the invention.

FIG. 2A shows an example result of amplification products obtained for target polynucleotides joined to adapter oligonucleotides, also referred to as “adapters,” according to methods of the invention.

FIG. 2B shows a side by side comparison of selected lanes from FIG. 2A, along with details about elements contained in the ligation reaction.

FIG. 3 shows a schematic drawing of one embodiment of the methods of the invention, with hairpin adapters comprising RNA at the 5′ end.

FIG. 4 shows a schematic drawing of one embodiment of the methods of the invention, with hairpin adapters comprising RNA at the 3′ end.

FIG. 5 shows a schematic drawing of one embodiment of the methods of the invention, with hairpin adapters comprising RNA at the 3′ end that are joined to a target polynucleotide, and further addition on non-hairpin adapters to ends of the target polynucleotide not joined to the hairpin adapter.

FIG. 6 shows a schematic drawing of one embodiment of the methods of the invention.

FIG. 7 shows various adapter designs, evaluated ligation efficiencies, and PCR amplified ligation products analyzed on an agarose gel.

FIG. 8 shows an agarose gel containing target polynucleotides, adapter oligonucleotides, and ligation products.

FIG. 9 shows an agarose gel containing PCR amplified ligation products.

FIG. 10 shows a schematic drawing of one embodiment of the methods of the invention.

### DEFINITIONS

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non limiting examples of polynucleotides: coding or non-coding regions of a gene or gene fragment, intergenic DNA, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), small nucleolar RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non nucleotide components. A polynucleotide may be further modified after polymerization, such as by conjugation with a labeling component. Polynucleotide sequences, when provided, are listed in the 5′ to 3′ direction, unless stated otherwise.

As used herein, the term “target polynucleotide” refers to a nucleic acid molecule or polynucleotide in a starting population of nucleic acid molecules having a target sequence whose presence, amount, and/or nucleotide sequence, or changes in these, are desired to be determined. In general, a target polynucleotide is a double-stranded nucleic acid molecule, and may be derived from any source of or process for generating double-stranded nucleic acid molecules.

As used herein, the term “target sequence” refers generally to a nucleic acid sequence on a single strand of nucleic acid. The target sequence may be a portion of a gene, a regulatory sequence, genomic DNA, cDNA, RNA including mRNA, miRNA, and rRNA, or others. The target sequence may be a target sequence from a sample or a secondary target such as a product of an amplification reaction.

A “nucleotide probe,” “probe,” or “tag oligonucleotide” refers to a polynucleotide used for detecting or identifying its corresponding target polynucleotide in a hybridization reaction. Thus, a tag oligonucleotide is hybridizable to one or more target polynucleotides. Tag oligonucleotides can be perfectly complementary to one or more target polynucleotides in a sample, or contain one or more nucleotides that are not complemented by a corresponding nucleotide in the one or more target polynucleotides in a sample.

“Hybridization” and “annealing” refer to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self hybridizing strand, or any combination of these. A hybridization reaction may constitute a step in a more extensive process, such as the initiation of a PCR, or the enzymatic cleavage of a polynucleotide by a ribozyme. A first sequence that can be stabilized via hydrogen bonding with the bases of the nucleotide residues of a second sequence is said to be “hybridizable” to said second sequence. In such a case, the second sequence can also be said to be hybridizable to the first sequence.

In general, a “complement” of a given sequence is a sequence that is fully complementary to and hybridizable to the given sequence. In general, a first sequence that is hybridizable to a second sequence or set of second sequences is specifically or selectively hybridizable to the second sequence or set of second sequences, such that hybridization to the second sequence or set of second sequences is preferred (e.g. thermodynamically more stable under a given set of conditions, such as stringent conditions commonly used in the art) to hybridization with non-target sequences during a hybridization reaction. Typically, hybridizable sequences share a degree of sequence complementarity over all or a portion of their respective lengths, such as between 25%-100% complementarity, including at least about 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, and 100% sequence complementarity.

The term “hybridized” as applied to a polynucleotide refers to a polynucleotide in a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self hybridizing strand, or any combination of these. The hybridization reaction may constitute a step in a more extensive process, such as the initiation of a PCR reaction, or the enzymatic cleavage of a polynucleotide by a ribozyme. A sequence hybridized with a given sequence is referred to as the “complement” of the given sequence.

As used herein, “expression” refers to the process by which a polynucleotide is transcribed into mRNA and/or the process by which the transcribed mRNA (also referred to as “transcript”) is subsequently being translated into peptides, polypeptides, or proteins. The transcripts and the encoded polypeptides are collectively referred to as “gene product.” If the polynucleotide is derived from genomic DNA, expression may include splicing of the mRNA in a eukaryotic cell.

### DETAILED DESCRIPTION OF THE INVENTION

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

In one aspect, the present invention provides a method for multiplex sequencing. In one embodiment, the method comprises sequencing a plurality of target polynucleotides in a single reaction chamber, wherein said target polynucleotides are from two or more different samples; and identifying the sample from which each of said sequenced target polynucleotides is derived with an accuracy of at least 95% based on a single barcode contained in the sequence of said target polynucleotide. Reaction chambers can be any suitable chamber known in the art for containing a sequencing reaction, non-limiting examples of which include tubes of various dimensions, wells of multi-well plates, and channels of flow cells. In some embodiments, the target polynucleotides comprise one or more sequences with which the sequencing reaction is calibrated. In some embodiments, the one or more sequences with which the sequencing reaction is calibrated are joined to the target polynucleotides prior to sequencing.

In another aspect, the invention provides a method of producing adapter-tagged target polynucleotides from a plurality of independent samples. In one embodiment, the method comprises: (a) providing a plurality of first adapter oligonucleotides, wherein each of said first adapter oligonucleotides comprises at least one of a plurality of barcode sequences, wherein each barcode sequence of the plurality of barcode sequences differs from every other barcode sequence in said plurality of barcode sequences at least three nucleotide positions; and (b) joining at least one of said first adapter oligonucleotides to said target polynucleotides of each of said samples, such that no barcode sequence is joined to said target polynucleotides of more than one of said samples. In some embodiments, the method further comprises (c) joining at least one of a plurality of second adapter oligonucleotides to said target polynucleotides of each of said samples from step (b), such that at least some of said target polynucleotides comprise said first adapter oligonucleotide at one end and said second adapter oligonucleotide at the other end. First and second adapter oligonucleotides can be the same or different, with different adapter oligonucleotides having different sequences and/or sequences of different lengths. A first adapter oligonucleotide can comprise one or more sequence regions that have the same sequence as one or more sequence regions of a second adapter oligonucleotide, and one or more sequence regions that have sequences that are different from one or more sequence regions of a second adapter oligonucleotide.

An adapter oligonucleotide includes any oligonucleotide having a sequence, at least a portion of which is known, that can be joined to a target polynucleotide. Adapter oligonucleotides can comprise DNA, RNA, nucleotide analogues, non-canonical nucleotides, labeled nucleotides, modified nucleotides, or combinations thereof. Adapter oligonucleotides can be single-stranded, double-stranded, or partial duplex. In general, a partial-duplex adapter comprises one or more single-stranded regions and one or more double-stranded regions. Double-stranded adapters can comprise two separate oligonucleotides hybridized to one another (also referred to as an “oligonucleotide duplex”), and hybridization may leave one or more blunt ends, one or more 3′ overhangs, one or more 5′ overhangs, one or more bulges resulting from mismatched and/or unpaired nucleotides, or any combination of these. In some embodiments, a single-stranded adapter comprises two or more sequences that are able to hybridize with one another. When two such hybridizable sequences are contained in a single-stranded adapter, hybridization yields a hairpin structure (hairpin adapter). When two hybridized regions of an adapter are separated from one another by a non-hybridized region, a “bubble” structure results. Adapters comprising a bubble structure can consist of a single adapter oligonucleotide comprising internal hybridizations, or may comprise two or more adapter oligonucleotides hybridized to one another. Internal sequence hybridization, such as between two hybridizable sequences in an adapter, can produce a double-stranded structure in a single-stranded adapter oligonucleotide. Adapters of different kinds can be used in combination, such as a hairpin adapter and a double-stranded adapter, or adapters of different sequences. Hybridizable sequences in a hairpin adapter may or may not include one or both ends of the oligonucleotide. When neither of the ends are included in the hybridizable sequences, both ends are “free” or “overhanging.” When only one end is hybridizable to another sequence in the adapter, the other end forms an overhang, such as a 3′ overhang or a 5′ overhang. When both the 5′-terminal nucleotide and the 3′-terminal nucleotide are included in the hybridizable sequences, such that the 5′-terminal nucleotide and the 3′-terminal nucleotide are complementary and hybridize with one another, the end is referred to as “blunt.” Different adapters can be joined to target polynucleotides in sequential reactions or simultaneously. For example, the first and second adapters can be added to the same reaction. Adapters can be manipulated prior to combining with target polynucleotides. For example, terminal phosphates can be added or removed.

In some embodiments, one of the hybridizable sequences in a single-stranded hairpin adapter comprises RNA. For example, an adapter can comprise a 5′ end comprising sequence A and a 3′ end comprising sequence A′, where A is hybridizable to A′, one of A or A′ comprises DNA, and the other of A or A′ comprises RNA. Similarly, an adapter can comprise a 5′ end comprising sequence B and a 3′ end comprising sequence B′, where B is hybridizable to B′, one of B or B′ comprises DNA, and the other of B or B′ comprises RNA. In some embodiments, one of A or A′ consists entirely of DNA, and/or one of A or A′ consists entirely of RNA. In some embodiment, one of B or B′ consists entirely of DNA, and/or one of B or B′ consists entirely of RNA. Sequence A can be the same as or different from sequence B and/or B′. Sequence A′ can be the same as or different from sequence B and/or B′. In some embodiments, the end of a hairpin comprising RNA (e.g. A, A′, B, or B′) further comprises one or more terminal DNA residues (e.g. 1, 2, 3, 4, 5, 6, 7, 8, 9, 10, 11, 12, 13, 14, 15, or more terminal DNA residues), such that the sequence comprising RNA is flanked by DNA residues at both ends (i.e. both the 5′ end and the 3′ end of the sequence comprising RNA). Hybridization of a sequence comprising RNA to a sequence comprising DNA creates an RNA-DNA heteroduplex. In some embodiments, RNA is cleaved by an enzyme that cleaves RNA from an RNA-DNA heteroduplex, such as enzymes comprising ribonuclease activity. Preferably, the enzyme comprising ribonuclease activity cleaves ribonucleotides in an RNA/DNA heteroduplex regardless of the identity and type of nucleotides adjacent to the ribonucleotide to be cleaved. It is preferred that the ribonuclease cleaves independent of sequence identity. Examples of suitable enzymes comprising ribonuclease activity for the methods and compositions of the invention are well known in the art, including ribonuclease H (RNase H) and enzymes comprising RNase H activity, e.g., Hybridase. In some embodiments, cleavage of RNA from an RNA-DNA heteroduplex removes all double-stranded character from a single-stranded hairpin adapter oligonucleotide, such that extension by a polymerase that uses the adapter as template requires no strand displacement step or strand displacement activity. In some embodiments, both ends of a hairpin adapter comprising one end comprising RNA are joined to a target polynucleotide, such that cleavage of the RNA from the RNA-DNA hetero duplex produces a 5′ overhang or a 3′ overhang. In some embodiments, an end comprising a 5′ overhang produced by cleavage of RNA from an RNA-DNA heteroduplex is filled in by the extension of the produced 3′ end using the 5′ overhang as template.

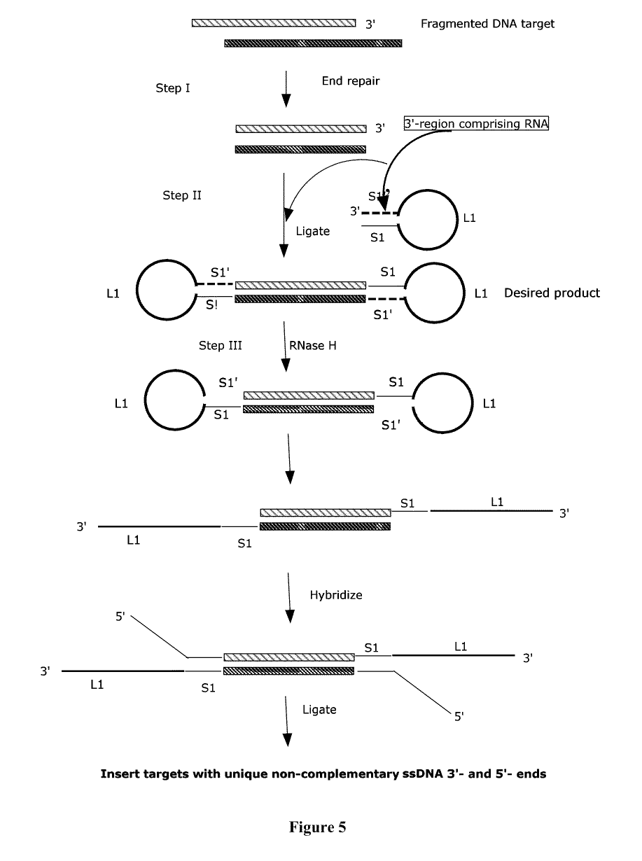

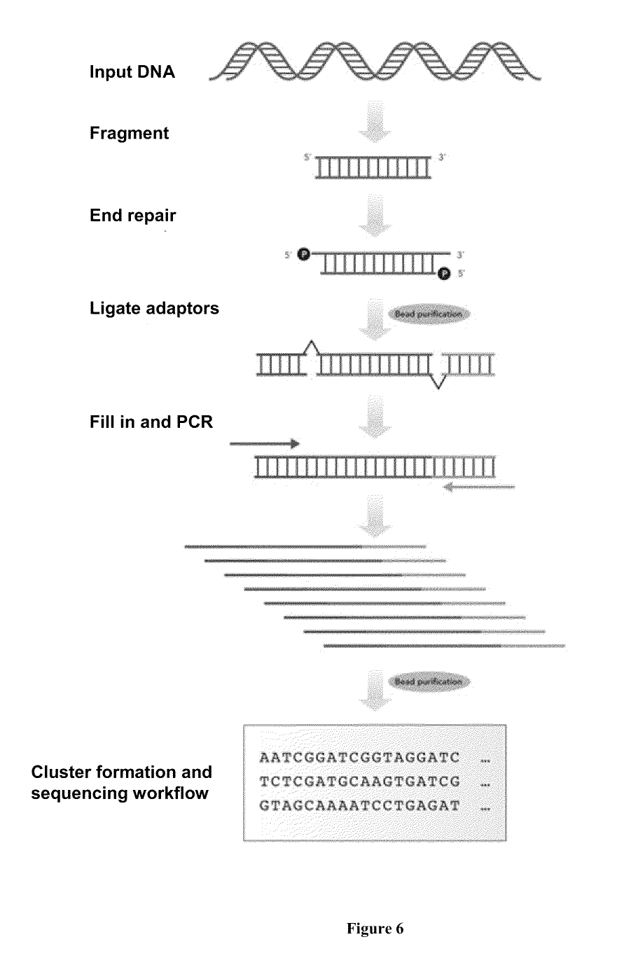

In some embodiments, where hairpin adapters comprising 3′ ends comprising RNA are joined to both 3′ ends of a double-stranded target polynucleotide, cleavage of RNA from the RNA-DNA heteroduplex is followed by hybridization of oligonucleotides to the adapter sequences joined in the first step, and ligation of the hybridized oligonucleotides to the 5′ ends of the double-stranded target polynucleotide to produce a target polynucleotide comprising non-complementary, single-stranded overhangs of both strands at both ends. Amplification of a double-stranded target polynucleotides comprising non-complementary, single-stranded overhangs on both strands at both ends can comprise the use of a first and second primer, wherein the first primer is hybridizable to one of the overhangs and the second primer is hybridizable to the complement of the overhang at the other end of the strand to which the first primer is hybridizable. Sequencing of double-stranded target polynucleotides comprising non-complementary, single-stranded overhangs on both strands at both ends can comprise the use of one or more sequencing primers hybridizable to one or more of the overhangs, or complements thereof. An illustrative example of the production of a double-stranded target polynucleotide comprising non-complementary, single-stranded overhangs on both strands at both ends is shown in FIG. 5.

Adapters can contain one or more of a variety of sequence elements, including but not limited to, one or more amplification primer annealing sequences or complements thereof, one or more sequencing primer annealing sequences or complements thereof, one or more barcode sequences, one or more common sequences shared among multiple different adapters or subsets of different adapters, one or more restriction enzyme recognition sites, one or more overhangs complementary to one or more target polynucleotide overhangs, one or more probe binding sites (e.g. for attachment to a sequencing platform, such as a flow cell for massive parallel sequencing, such as developed by Illumina, Inc.), one or more random or near-random sequences (e.g. one or more nucleotides selected at random from a set of two or more different nucleotides at one or more positions, with each of the different nucleotides selected at one or more positions represented in a pool of adapters comprising the random sequence), and combinations thereof. Two or more sequence elements can be non-adjacent to one another (e.g. separated by one or more nucleotides), adjacent to one another, partially overlapping, or completely overlapping. For example, an amplification primer annealing sequence can also serve as a sequencing primer annealing sequence. Sequence elements can be located at or near the 3′ end, at or near the 5′ end, or in the interior of the adapter oligonucleotide. When an adapter oligonucleotide is capable of forming secondary structure, such as a hairpin, sequence elements can be located partially or completely outside the secondary structure, partially or completely inside the secondary structure, or in between sequences participating in the secondary structure. For example, when an adapter oligonucleotide comprises a hairpin structure, sequence elements can be located partially or completely inside or outside the hybridizable sequences (the “stem”), including in the sequence between the hybridizable sequences (the “loop”). In some embodiments, the first adapter oligonucleotides in a plurality of first adapter oligonucleotides having different barcode sequences comprise a sequence element common among all first adapter oligonucleotides in the plurality. In some embodiments, all second adapter oligonucleotides comprise a sequence element common among all second adapter oligonucleotides that is different from the common sequence element shared by the first adapter oligonucleotides. A difference in sequence elements can be any such that at least a portion of different adapters do not completely align, for example, due to changes in sequence length, deletion or insertion of one or more nucleotides, or a change in the nucleotide composition at one or more nucleotide positions (such as a base change or base modification). In some embodiments, an adapter oligonucleotide comprises a 5′ overhang, a 3′ overhang, or both that is complementary to one or more target polynucleotides. Complementary overhangs can be one or more nucleotides in length, including but not limited to 1, 2, 3, 4, 5, 6, 7, 8, 9, 10, 11, 12, 13, 14, 15, or more nucleotides in length. Complementary overhangs may comprise a fixed sequence. Complementary overhangs may comprise a random sequence of one or more nucleotides, such that one or more nucleotides are selected at random from a set of two or more different nucleotides at one or more positions, with each of the different nucleotides selected at one or more positions represented in a pool of adapters with complementary overhangs comprising the random sequence. In some embodiments, an adapter overhang is complementary to a target polynucleotide overhang produced by restriction endonuclease digestion. In some embodiments, an adapter overhang consists of an adenine or a thymine.

In some embodiments, one or more of the adapter oligonucleotides comprises SEQ ID NO: 1. In some embodiments, one or more of the adapter oligonucleotides comprises SEQ ID NO: 2. In some embodiments, the sequence element common among all first adapter oligonucleotides comprises SEQ ID NO: 1 or SEQ ID NO: 2. In some embodiments, the sequence element common among all second adapter oligonucleotides comprises SEQ ID NO: 1 or SEQ ID NO: 2. In some embodiments, one of SEQ ID NO: 1 or SEQ ID NO: 2 is common among all first adapter oligonucleotides and the other of SEQ ID NO: 1 or SEQ ID NO: 2 is common among all second adapter oligonucleotides. In some embodiments, one or more of the adapter oligonucleotides comprises SEQ ID NO: 3. In some embodiments, one or more of the adapter oligonucleotides comprises SEQ ID NO: 4. In some embodiments the 3′-most nucleotide of SEQ ID NO: 3 and/or SEQ ID NO: 4 is followed by one or more nucleotides of a barcode sequence.

In some embodiments, an adapter comprising an oligonucleotide duplex comprises an oligonucleotide comprising SEQ ID NO: 86 and/or an oligonucleotide comprising SEQ ID NO: 87. In some embodiments, an adapter comprising an oligonucleotide duplex comprises an oligonucleotide comprising SEQ ID NO: 88, and/or an oligonucleotide comprising SEQ ID NO: 89.

Adapter oligonucleotides can have any suitable length, at least sufficient to accommodate the one or more sequence elements of which they are comprised. In some embodiments, adapters are about, less than about, or more than about 10, 15, 20, 25, 30, 35, 40, 45, 50, 55, 60, 65, 70, 75, 80, 90, 100, 200, or more nucleotides in length. In some embodiments, the stem of a hairpin adapter is about, less than about, or more than about 1, 2, 3, 4, 5, 6, 7, 8, 9, 10, 11, 12, 13, 14, 15, 20, 25, 30, 35, 40, 45, 50, 75, 100, or more nucleotides in length. Stems may be designed using a variety of different sequences that result in hybridization between the complementary regions on a hairpin adapter, resulting in a local region of double-stranded DNA. For example, stem sequences may be utilized that are from 15 to 18 nucleotides in length with equal representation of G:C and A:T base pairs. Such stem sequences are predicted to form stable dsDNA structures below their predicted melting temperatures of ˜45° C. Sequences participating in the stem of the hairpin can be perfectly complementary, such that each base of one region in the stem hybridizes via hydrogen bonding with each base in the other region in the stem according to Watson-Crick base-pairing rules. Alternatively, sequences in the stem may deviate from perfect complementarity. For example, there can be mismatches and or bulges within the stem structure created by opposing bases that do not follow Watson-Crick base pairing rules, and/or one or more nucleotides in one region of the stem that do not have the one or more corresponding base positions in the other region participating in the stem. Mismatched sequences may be cleaved using enzymes that recognize mismatches. The stem of a hairpin can comprise DNA, RNA, or both DNA and RNA. In some embodiments, the stem and/or loop of a hairpin, or one or both of the hybridizable sequences forming the stem of a hairpin, comprise nucleotides, bonds, or sequences that are substrates for cleavage, such as by an enzyme, including but not limited to endonucleases and glycosylases. The composition of a stem may be such that only one of the hybridizable sequences forming the stem is cleaved. For example, one of the sequences forming the stem may comprise RNA while the other sequence forming the stem consists of DNA, such that cleavage by an enzyme that cleaves RNA in an RNA-DNA duplex, such as RNase H, cleaves only the sequence comprising RNA. The stem and/or loop of a hairpin can comprise non-canonical nucleotides (e.g. uracil), and/or methylated nucleotides. In some embodiments, one strand of a hairpin adapter stem comprises SEQ ID NO: 1 or SEQ ID NO: 2. In some embodiments, the loop sequence of a hairpin adapter is about, less than about, or more than about 5, 10, 15, 20, 25, 30, 35, 40, 45, 50, or more nucleotides in length.

As used herein, the term “barcode” refers to a known nucleic acid sequence that allows some feature of a polynucleotide with which the barcode is associated to be identified. In some embodiments, the feature of the polynucleotide to be identified is the sample from which the polynucleotide is derived. In some embodiments, barcodes are at least 3, 4, 5, 6, 7, 8, 9, 10, 11, 12, 13, 14, 15, or more nucleotides in length. In some embodiments, barcodes are shorter than 10, 9, 8, 7, 6, 5, or 4 nucleotides in length. In some embodiments, barcodes associated with some polynucleotides are of different length than barcodes associated with other polynucleotides. In general, barcodes are of sufficient length and comprise sequences that are sufficiently different to allow the identification of samples based on barcodes with which they are associated. In some embodiments, a barcode, and the sample source with which it is associated, can be identified accurately after the mutation, insertion, or deletion of one or more nucleotides in the barcode sequence, such as the mutation, insertion, or deletion of 1, 2, 3, 4, 5, 6, 7, 8, 9, 10, or more nucleotides. In some embodiments, each barcode in a plurality of barcodes differ from every other barcode in the plurality at least three nucleotide positions, such as at least 3, 4, 5, 6, 7, 8, 9, 10, or more positions. In some embodiments, both the first adapter and the second adapter comprise at least one of a plurality of barcode sequences. In some embodiments, barcodes for second adapter oligonucleotides are selected independently from barcodes for first adapter oligonucleotides. In some embodiments, first adapter oligonucleotides and second adapter oligonucleotides having barcodes are paired, such that adapters of the pair comprise the same or different one or more barcodes. In some embodiments, the methods of the invention further comprise identifying the sample from which a target polynucleotide is derived based on a barcode sequence to which the target polynucleotide is joined. In general, a barcode comprises a nucleic acid sequence that when joined to a target polynucleotide serves as an identifier of the sample from which the target polynucleotide was derived.

In some embodiments, the plurality of barcode sequences from which barcode sequences are selected includes sequences selected from the group consisting of: AAA, TTT, CCC, GGG. In some embodiments, the plurality of barcode sequences from which barcode sequences are selected includes sequences selected from the group consisting of: AAAA, CTGC, GCTG, TGCT, ACCC, CGTA, GAGT, TTAG, AGGG, CCAT, GTCA, TATC, ATTT, CACG, GGAC, and TCGA. In some embodiments, the plurality of barcode sequences from which barcode sequences are selected includes sequences selected from the group consisting of: AAAAA, AACCC, AAGGG, AATTT, ACACG, ACCAT, ACGTA, ACTGC, AGAGT, AGCTG, AGGAC, AGTCA, ATATC, ATCGA, ATGCT, ATTAG, CAACT, CACAG, CAGTC, CATGA, CCAAC, CCCCA, CCGGT, CCTTG, CGATA, CGCGC, CGGCG, CGTAT, CTAGG, CTCTT, CTGAA, CTTCC, GAAGC, GACTA, GAGAT, GATCG, GCATT, GCCGG, GCGCC, GCTAA, GGAAG, GGCCT, GGGGA, GGTTC, GTACA, GTCAC, GTGTG, GTTTT, TAATG, TACGT, TAGCA, TATAC, TCAGA, TCCTC, TCGAG, TCTCT, TGACC, TGCAA, TGGTT, TGTGG, TTAAT, TTCCG, TTGGC, and TTTTA.

The terms “joining” and “ligation” as used herein, with respect to two polynucleotides, such as an adapter oligonucleotide and a target polynucleotide, refers to the covalent attachment of two separate polynucleotides to produce a single larger polynucleotide with a contiguous backbone. Methods for joining two polynucleotides are known in the art, and include without limitation, enzymatic and non-enzymatic (e.g. chemical) methods. Examples of ligation reactions that are non-enzymatic include the non-enzymatic ligation techniques described in U.S. Pat. Nos. 5,780,613 and 5,476,930, which are herein incorporated by reference. In some embodiments, an adapter oligonucleotide is joined to a target polynucleotide by a ligase, for example a DNA ligase or RNA ligase. Multiple ligases, each having characterized reaction conditions, are known in the art, and include, without limitation NAD⁺-dependent ligases including tRNA ligase, Taq DNA ligase, Thermus filiformis DNA ligase, Escherichia coli DNA ligase, Tth DNA ligase, Thermus scotoductus DNA ligase (I and II), thermostable ligase, Ampligase thermostable DNA ligase, VanC-type ligase, 9° N DNA Ligase, Tsp DNA ligase, and novel ligases discovered by bioprospecting; ATP-dependent ligases including T4 RNA ligase, T4 DNA ligase, T3 DNA ligase, T7 DNA ligase, Pfu DNA ligase, DNA ligase 1, DNA ligase III, DNA ligase IV, and novel ligases discovered by bioprospecting; and wild-type, mutant isoforms, and genetically engineered variants thereof. Ligation can be between polynucleotides having hybridizable sequences, such as complementary overhangs. Ligation can also be between two blunt ends. Generally, a 5′ phosphate is utilized in a ligation reaction. The 5′ phosphate can be provided by the target polynucleotide, the adapter oligonucleotide, or both. 5′ phosphates can be added to or removed from polynucleotides to be joined, as needed. Methods for the addition or removal of 5′ phosphates are known in the art, and include without limitation enzymatic and chemical processes. Enzymes useful in the addition and/or removal of 5′ phosphates include kinases, phosphatases, and polymerases. In some embodiments, both of the two ends joined in a ligation reaction (e.g. an adapter end and a target polynucleotide end) provide a 5′ phosphate, such that two covalent linkages are made in joining the two ends. In some embodiments, only one of the two ends joined in a ligation reaction (e.g. only one of an adapter end and a target polynucleotide end) provides a 5′ phosphate, such that only one covalent linkage is made in joining the two ends. In some embodiments, only one strand at one or both ends of a target polynucleotide is joined to an adapter oligonucleotide. In some embodiments, both strands at one or both ends of a target polynucleotide are joined to an adapter oligonucleotide. In some embodiments, 3′ phosphates are removed prior to ligation. In some embodiments, an adapter oligonucleotide is added to both ends of a target polynucleotide, wherein one or both strands at each end are joined to one or more adapter oligonucleotides. When both strands at both ends are joined to an adapter oligonucleotide, joining can be followed by a cleavage reaction that leaves a 5′ overhang that can serve as a template for the extension of the corresponding 3′ end, which 3′ end may or may not include one or more nucleotides derived from the adapter oligonucleotide. In some embodiments, a target polynucleotide is joined to a first adapter oligonucleotide on one end and a second adapter oligonucleotide on the other end. In some embodiments, the target polynucleotide and the adapter to which it is joined comprise blunt ends. In some embodiments, separate ligation reactions are carried out for each sample, using a different first adapter oligonucleotide comprising at least one barcode sequence for each sample, such that no barcode sequence is joined to the target polynucleotides of more than one sample. A target polynucleotide that has an adapter oligonucleotide joined to it is considered “tagged” by the joined adapter.

In some embodiments, joining of an adapter to a target polynucleotide produces a joined product polynucleotide having a 3′ overhang comprising a nucleotide sequence derived from the adapter. In some embodiments, a primer oligonucleotide comprising a sequence complementary to all or a portion of the 3′ overhang is hybridized to the overhang and extended using a DNA polymerase to produce a primer extension product hybridized to one strand of the joined product polynucleotide. The DNA polymerase may comprise strand displacement activity, such that one strand of the joined product polynucleotide is displaced during primer extension.

In some embodiments, after joining at least one adapter oligonucleotide to a target polynucleotide, the 3′ end of one or more target polynucleotides is extended using the one or more joined adapter oligonucleotides as template. For example, an adapter comprising two hybridized oligonucleotides that is joined to only the 5′ end of a target polynucleotide allows for the extension of the unjoined 3′ end of the target using the joined strand of the adapter as template, concurrently with or following displacement of the unjoined strand. If both strands of an adapter comprising two hybridized oligonucleotides are joined to a target polynucleotide such that the joined product has a 5′ overhang, the complementary 3′ end can be extended using the 5′ overhang as template. As a further example, a hairpin adapter oligonucleotide can be joined to the 5′ end of a target polynucleotide. While double-stranded in secondary structure, such a hairpin adapter remains single-stranded, and is thus a 5′ overhang appended to the target polynucleotide (e.g. when the 5′ end of the hairpin adapter is not joined to the target polynucleotide). Removal of the secondary structure, either prior to (e.g. thermal denaturing, or degradation) or concurrently with (e.g. strand displacement) the activity of a polymerase, provides a template for the extension of the 3′ end of the complementary strand of the target polynucleotide. In some embodiments, the 3′ end of the target polynucleotide that is extended comprises one or more nucleotides from an adapter oligonucleotide. For target polynucleotides to which adapters are joined on both ends, extension can be carried out for both 3′ ends of a double-stranded target polynucleotide having 5′ overhangs. This 3′ end extension, or “fill-in” reaction, generates a complementary sequence, or “complement,” to the adapter oligonucleotide template that is hybridized to the template, thus filling in the 5′ overhang to produce a double-stranded sequence region. Where both ends of a double-stranded target polynucleotide have 5′ overhangs that are filled in by extension of the complementary strands' 3′ ends, the product is completely double-stranded. Extension can be carried out by any suitable polymerase known in the art, such as a DNA polymerase, many of which are commercially available. DNA polymerases can comprise DNA-dependent DNA polymerase activity, RNA-dependent DNA polymermase activity, or DNA-dependent and RNA-dependent DNA polymerase activity. DNA polymerases can be thermostable or non-thermostable. Examples of DNA polymerases include, but are not limited to, Taq polymerase, Tth polymerase, Tli polymerase, Pfu polymerase, Pfutubo polymerase, Pyrobest polymerase, Pwo polymerase, KOD polymerase, Bst polymerase, Sac polymerase, Sso polymerase, Poc polymerase, Pab polymerase, Mth polymerase, Pho polymerase, ES4 polymerase, VENT polymerase, DEEPVENT polymerase, EX-Taq polymerase, LA-Taq polymerase, Expand polymerases, Platinum Taq polymerases, Hi-Fi polymerase, Tbr polymerase, Tfl polymerase, Tru polymerase, Tac polymerase, Tne polymerase, Tma polymerase, Tih polymerase, Tfi polymerase, Klenow fragment, and variants, modified products and derivatives thereof 3′ end extension can be performed before or after pooling of target polynucleotides from independent samples.

In some embodiments, the fill-in reaction is followed by or performed as part of amplification of one or more target polynucleotides using a first primer and a second primer, wherein the first primer comprises a sequence that is hybridizable to at least a portion of the complement of one or more of the first adapter oligonucleotides, and further wherein the second primer comprises a sequence that is hybridizable to at least a portion of the complement of one or more of the second adapter oligonucleotides. Each of the first and second primers may be of any suitable length, such as about, less than about, or more than about 10, 15, 20, 25, 30, 35, 40, 45, 50, 55, 60, 65, 70, 75, 80, 90, 100, or more nucleotides, any portion or all of which may be complementary to the corresponding target sequence (e.g. about, less than about, or more than about 5, 10, 15, 20, 25, 30, 35, 40, 45, 50, or more nucleotides). “Amplification” refers to any process by which the copy number of a target sequence is increased. Methods for primer-directed amplification of target polynucleotides are known in the art, and include without limitation, methods based on the polymerase chain reaction (PCR). Conditions favorable to the amplification of target sequences by PCR are known in the art, can be optimized at a variety of steps in the process, and depend on characteristics of elements in the reaction, such as target type, target concentration, sequence length to be amplified, sequence of the target and/or one or more primers, primer length, primer concentration, polymerase used, reaction volume, ratio of one or more elements to one or more other elements, and others, some or all of which can be altered. In general, PCR involves the steps of denaturation of the target to be amplified (if double stranded), hybridization of one or more primers to the target, and extension of the primers by a DNA polymerase, with the steps repeated (or “cycled”) in order to amplify the target sequence. Steps in this process can be optimized for various outcomes, such as to enhance yield, decrease the formation of spurious products, and/or increase or decrease specificity of primer annealing. Methods of optimization are well known in the art and include adjustments to the type or amount of elements in the amplification reaction and/or to the conditions of a given step in the process, such as temperature at a particular step, duration of a particular step, and/or number of cycles. In some embodiments, an amplification reaction comprises at least 5, 10, 15, 20, 25, 30, 35, 50, or more cycles. In some embodiments, an amplification reaction comprises no more than 5, 10, 15, 20, 25, 35, 50, or more cycles. Cycles can contain any number of steps, such as 1, 2, 3, 4, 5, 6, 7, 8, 9, 10 or more steps. Steps can comprise any temperature or gradient of temperatures, suitable for achieving the purpose of the given step, including but not limited to, 3′ end extension (e.g. adapter fill-in), primer annealing, primer extension, and strand denaturation. Steps can be of any duration, including but not limited to about, less than about, or more than about 1, 5, 10, 15, 20, 25, 30, 35, 40, 45, 50, 55, 60, 70, 80, 90, 100, 120, 180, 240, 300, 360, 420, 480, 540, 600, or more seconds, including indefinitely until manually interrupted. Cycles of any number comprising different steps can be combined in any order. In some embodiments, different cycles comprising different steps are combined such that the total number of cycles in the combination is about, less that about, or more than about 5, 10, 15, 20, 25, 30, 35, 50, or more cycles. In some embodiments, one or more of the primers comprises SEQ ID NO: 1. In some embodiments, one or more of the primers comprises SEQ ID NO: 2. In some embodiments, amplification is performed following the fill-in reaction. Amplification can be performed before or after pooling of target polynucleotides from independent samples.

In some embodiments, target polynucleotides from independent samples are pooled after the joining step. Pooling can be performed immediately after the joining step, or following one or more intervening steps between joining and pooling. Pools can comprise any fraction of the total target polynucleotides from a joining reaction, including the whole reaction volume. Samples can be pooled evenly or unevenly. Target polynucleotides can be further processed before or after pooling, for example to purify desired products or eliminate undesired products. Pools can comprise polynucleotides from any number of independent samples, such as at least 2, 3, 4, 5, 6, 7, 8, 9, 10, 12, 16, 20, 24, 28, 32, 36, 40, 50, 60, 70, 80, 90, 100, 128, 192, 384, 500, 1000 or more samples. In some embodiments, target polynucleotides are pooled based on the barcodes to which they are joined. In some embodiments, target polynucleotides from independent samples are pooled such that all four bases are evenly represented at one or more positions along the barcode, among barcodes included in the pool. In some embodiments, target polynucleotides from independent samples are pooled such that all four bases are evenly represented at every position along the barcode, among barcodes included in the pool. Where only one barcode is joined to polynucleotides of each sample, samples can be pooled in multiples of four in order to represent all four bases at one or more positions along the barcode evenly, for example 4, 8, 12, 16, 20, 24, 28, 32, 36, 40, 44, 48, 52, 56, 60, 64, 96, 128, 192, 256, 384, and so on. Where two barcodes are included in the joining reaction for each sample, such as two different first adapter oligonucleotides or one first adapter oligonucleotide and one second adapter oligonucleotide each having barcodes, samples can be pooled in multiples of two in order to evenly represent all four bases at one or more positions along the barcode, for example 2, 4, 6, 8, 10, 12, 14, 16, 18, 20, 22, 24, 48, 64, 96, 128, 256, 384, and so on. All combinations of the number of barcodes included in the joining reaction for target polynucleotides from each sample and the multiples in which samples are pooled in order to evenly represent all four nucleotides at one or more positions along the barcode are contemplated by the methods of the invention.

In some embodiments, pooling of target polynucleotides is followed by sequencing one or more polynucleotides in the pool. Sequencing processes are generally template dependent. Nucleic acid sequence analysis that employs template dependent synthesis identifies individual bases, or groups of bases as they are added during a template mediated synthesis reaction, such as a primer extension reaction, where the identity of the base is complementary to the template sequence to which the primer sequence is hybridized during synthesis. Other such processes include ligation driven processes, where oligonucleotides or polynucleotides are complexed with an underlying template sequence, in order to identify the sequence of nucleotides in that sequence. Typically, such processes are enzymatically mediated using nucleic acid polymerases, such as DNA polymerases, RNA polymerases, reverse transcriptases, and the like, or other enzymes such as in the case of ligation driven processes, e.g., ligases.

Sequence analysis using template dependent synthesis can include a number of different processes. For example, in the ubiquitously practiced four-color Sanger sequencing methods, a population of template molecules is used to create a population of complementary fragment sequences. Primer extension is carried out in the presence of the four naturally occurring nucleotides, and with a sub-population of dye labeled terminator nucleotides, e.g., dideoxyribonucleotides, where each type of terminator (ddATP, ddGTP, ddTTP, ddCTP) includes a different detectable label. As a result, a nested set of fragments is created where the fragments terminate at each nucleotide in the sequence beyond the primer, and are labeled in a manner that permits identification of the terminating nucleotide. The nested fragment population is then subjected to size based separation, e.g., using capillary electrophoresis, and the labels associated with each different sized fragment is identified to identify the terminating nucleotide. As a result, the sequence of labels moving past a detector in the separation system provides a direct readout of the sequence information of the synthesized fragments, and by complementarity, the underlying template (See, e.g., U.S. Pat. No. 5,171,534, incorporated herein by reference in its entirety for all purposes).

Other examples of template dependent sequencing methods include sequence by synthesis processes, where individual nucleotides are identified iteratively, as they are added to the growing primer extension product.

Pyrosequencing is an example of a sequence by synthesis process that identifies the incorporation of a nucleotide by assaying the resulting synthesis mixture for the presence of by-products of the sequencing reaction, namely pyrophosphate. In particular, a primer/template/polymerase complex is contacted with a single type of nucleotide. If that nucleotide is incorporated, the polymerization reaction cleaves the nucleoside triphosphate between the α and β phosphates of the triphosphate chain, releasing pyrophosphate. The presence of released pyrophosphate is then identified using a chemiluminescent enzyme reporter system that converts the pyrophosphate, with AMP, into ATP, then measures ATP using a luciferase enzyme to produce measurable light signals. Where light is detected, the base is incorporated, where no light is detected, the base is not incorporated. Following appropriate washing steps, the various bases are cyclically contacted with the complex to sequentially identify subsequent bases in the template sequence. See, e.g., U.S. Pat. No. 6,210,891, incorporated herein by reference in its entirety for all purposes).

In related processes, the primer/template/polymerase complex is immobilized upon a substrate and the complex is contacted with labeled nucleotides. The immobilization of the complex may be through the primer sequence, the template sequence and/or the polymerase enzyme, and may be covalent or noncovalent. For example, immobilization of the complex can be via a linkage between the polymerase or the primer and the substrate surface. A variety of types of linkages are useful for this attachment, including, e.g., provision of biotinylated surface components, using e.g., biotin-PEG-silane linkage chemistries, followed by biotinylation of the molecule to be immobilized, and subsequent linkage through, e.g., a streptavidin bridge. Other synthetic coupling chemistries, as well as non-specific protein adsorption can also be employed for immobilization. In alternate configurations, the nucleotides are provided with and without removable terminator groups. Upon incorporation, the label is coupled with the complex and is thus detectable. In the case of terminator bearing nucleotides, all four different nucleotides, bearing individually identifiable labels, are contacted with the complex. Incorporation of the labeled nucleotide arrests extension, by virtue of the presence of the terminator, and adds the label to the complex. The label and terminator are then removed from the incorporated nucleotide, and following appropriate washing steps, the process is repeated. In the case of non-terminated nucleotides, a single type of labeled nucleotide is added to the complex to determine whether it will be incorporated, as with pyrosequencing. Following removal of the label group on the nucleotide and appropriate washing steps, the various different nucleotides are cycled through the reaction mixture in the same process. See, e.g., U.S. Pat. No. 6,833,246, incorporated herein by reference in its entirety for all purposes). For example, the Illumina Genome Analyzer System is based on technology described in WO 98/44151, hereby incorporated by reference, wherein DNA molecules are bound to a sequencing platform (flow cell) via an anchor probe binding site (otherwise referred to as a flow cell binding site) and amplified in situ on a glass slide. The DNA molecules are then annealed to a sequencing primer and sequenced in parallel base-by-base using a reversible terminator approach. Typically, the Illumina Genome Analyzer System utilizes flow-cells with 8 channels, generating sequencing reads of 18 to 36 bases in length, generating >1.3 Gbp of high quality data per run (see www.illumina.com).

In yet a further sequence by synthesis process, the incorporation of differently labeled nucleotides is observed in real time as template dependent synthesis is carried out. In particular, an individual immobilized primer/template/polymerase complex is observed as fluorescently labeled nucleotides are incorporated, permitting real time identification of each added base as it is added. In this process, label groups are attached to a portion of the nucleotide that is cleaved during incorporation. For example, by attaching the label group to a portion of the phosphate chain removed during incorporation, i.e., a β,γ, or other terminal phosphate group on a nucleoside polyphosphate, the label is not incorporated into the nascent strand, and instead, natural DNA is produced. Observation of individual molecules typically involves the optical confinement of the complex within a very small illumination volume. By optically confining the complex, one creates a monitored region in which randomly diffusing nucleotides are present for a very short period of time, while incorporated nucleotides are retained within the observation volume for longer as they are being incorporated. This results in a characteristic signal associated with the incorporation event, which is also characterized by a signal profile that is characteristic of the base being added. In related aspects, interacting label components, such as fluorescent resonant energy transfer (FRET) dye pairs, are provided upon the polymerase or other portion of the complex and the incorporating nucleotide, such that the incorporation event puts the labeling components in interactive proximity, and a characteristic signal results, that is again, also characteristic of the base being incorporated (See, e.g., U.S. Pat. Nos. 6,056,661, 6,917,726, 7,033,764, 7,052,847, 7,056,676, 7,170,050, 7,361,466, 7,416,844 and Published U.S. Patent Application No. 2007-0134128, the full disclosures of which are hereby incorporated herein by reference in their entirety for all purposes).

In some embodiments, the nucleic acids in the sample can be sequenced by ligation. This method uses a DNA ligase enzyme to identify the target sequence, for example, as used in the polony method and in the SOLiD technology (Applied Biosystems, now Invitrogen). In general, a pool of all possible oligonucleotides of a fixed length is provided, labeled according to the sequenced position. Oligonucleotides are annealed and ligated; the preferential ligation by DNA ligase for matching sequences results in a signal corresponding to the complementary sequence at that position.

In some embodiments, sequencing comprises extension of a sequencing primer comprising a sequence hybridizable to at least a portion of the complement of the first adapter oligonucleotide. In some embodiments, sequencing comprises extension of a sequencing primer comprising a sequence hybridizable to at least a portion of the complement of the second adapter oligonucleotide. A sequencing primer may be of any suitable length, such as about, less than about, or more than about 10, 15, 20, 25, 30, 35, 40, 45, 50, 55, 60, 65, 70, 75, 80, 90, 100, or more nucleotides, any portion or all of which may be complementary to the corresponding target sequence (e.g. about, less than about, or more than about 5, 10, 15, 20, 25, 30, 35, 40, 45, 50, or more nucleotides). In some embodiments, the sequencing primer comprises SEQ ID NO: 1 or SEQ ID NO:2. In some embodiments, the sequencing primer comprises SEQ ID NO: 5. In some embodiments, the sequencing primer comprises SEQ ID NO: 6. In some embodiments, sequencing comprises a calibration step, wherein the calibration is based on each of the nucleotides at one or more nucleotide positions in the barcode sequences. Calibration can be useful in processing the sequencing data, for example, by facilitating or increasing the accuracy of identifying a base at a given position in the sequence.

In some embodiments, accurate identification of the sample from which a target polynucleotide is derived is based on at least a portion of the sequence obtained for the target polynucleotide and is at least 90%, 95%, 96%, 97%, 98%, 99%, 99.5%, 99.8%, 99.85%, 99.9%, 99.95%, 99.99%, or more accurate. In some embodiments, the sample source of a target polynucleotide is identified based on a single barcode contained in the sequence. In some embodiments, accuracy can be increased by identifying the source of a target polynucleotide using two or more barcodes contained in the sequence. Multiple barcodes can be joined to a target polynucleotide by the incorporation of multiple barcodes into a single adapter to which a target polynucleotide is joined, and/or by joining two or more adapters having one or more barcodes to a target polynucleotide. In some embodiments, the identity of the sample source of a target polynucleotide comprising two or more barcode sequences may be accurately determined using only one of the barcode sequences that it comprises. In general, accurate identification of a sample from which a target polynucleotide is derived comprises correct identification of a sample source from among two or more samples in a pool, such as about, less than about, or more than about 2, 3, 4, 5, 6, 7, 8, 9, 10, 12, 16, 20, 24, 28, 32, 36, 40, 50, 60, 70, 80, 90, 100, 128, 192, 384, 500, 1000 or more samples in a pool.

The different samples from which the target polynucleotides are derived can comprise multiple samples from the same individual, samples from different individuals, or combinations thereof. In some embodiments, a sample comprises a plurality of polynucleotides from a single individual. In some embodiments, a sample comprises a plurality of polynucleotides from two or more individuals. An individual is any organism or portion thereof from which target polynucleotides can be derived, non-limiting examples of which include plants, animals, fungi, protists, monerans, viruses, mitochondria, and chloroplasts. Sample polynucleotides can be isolated from a subject, such as a cell sample, tissue sample, or organ sample derived therefrom, including, for example, cultured cell lines, biopsy, blood sample, or fluid sample containing a cell. The subject may be an animal, including but not limited to, an animal such as a cow, a pig, a mouse, a rat, a chicken, a cat, a dog, etc., and is usually a mammal, such as a human. Samples can also be artificially derived, such as by chemical synthesis. In some embodiments, the samples comprise DNA. In some embodiments, the samples comprise genomic DNA. In some embodiments, the samples comprise mitochondrial DNA, chloroplast DNA, plasmid DNA, bacterial artificial chromosomes, yeast artificial chromosomes, oligonucleotide tags, or combinations thereof. In some embodiments, the samples comprise DNA generated by primer extension reactions using any suitable combination of primers and a DNA polymerase, including but not limited to polymerase chain reaction (PCR), reverse transcription, and combinations thereof. Where the template for the primer extension reaction is RNA, the product of reverse transcription is referred to as complementary DNA (cDNA). Primers useful in primer extension reactions can comprise sequences specific to one or more targets, random sequences, partially random sequences, and combinations thereof. Reaction conditions suitable for primer extension reactions are known in the art. In general, sample polynucleotides comprise any polynucleotide present in a sample, which may or may not include target polynucleotides.

Methods for the extraction and purification of nucleic acids are well known in the art. For example, nucleic acids can be purified by organic extraction with phenol, phenol/chloroform/isoamyl alcohol, or similar formulations, including TRIzol and TriReagent. Other non-limiting examples of extraction techniques include: (1) organic extraction followed by ethanol precipitation, e.g., using a phenol/chloroform organic reagent (Ausubel et al., 1993), with or without the use of an automated nucleic acid extractor, e.g., the Model 341 DNA Extractor available from Applied Biosystems (Foster City, Calif.); (2) stationary phase adsorption methods (U.S. Pat. No. 5,234,809; Walsh et al., 1991); and (3) salt-induced nucleic acid precipitation methods (Miller et al., (1988), such precipitation methods being typically referred to as “salting-out” methods. Another example of nucleic acid isolation and/or purification includes the use of magnetic particles to which nucleic acids can specifically or non-specifically bind, followed by isolation of the beads using a magnet, and washing and eluting the nucleic acids from the beads (see e.g. U.S. Pat. No. 5,705,628). In some embodiments, the above isolation methods may be preceded by an enzyme digestion step to help eliminate unwanted protein from the sample, e.g., digestion with proteinase K, or other like proteases. See, e.g., U.S. Pat. No. 7,001,724. If desired, RNase inhibitors may be added to the lysis buffer. For certain cell or sample types, it may be desirable to add a protein denaturation/digestion step to the protocol. Purification methods may be directed to isolate DNA, RNA, or both. When both DNA and RNA are isolated together during or subsequent to an extraction procedure, further steps may be employed to purify one or both separately from the other. Sub-fractions of extracted nucleic acids can also be generated, for example, purification by size, sequence, or other physical or chemical characteristic. In addition to an initial nucleic isolation step, purification of nucleic acids can be performed after any step in the methods of the invention, such as to remove excess or unwanted reagents, reactants, or products.

In some embodiments, sample polynucleotides are fragmented into a population of fragmented insert DNA molecules of one or more specific size range(s). In some embodiments, fragments are generated from at least about 1, 10, 100, 1000, 10000, 100000, 300000, 500000, or more genome-equivalents of starting DNA. Fragmentation may be accomplished by methods known in the art, including chemical, enzymatic, and mechanical fragmentation. In some embodiments, the fragments have an average length from about 10 to about 10,000 nucleotides. In some embodiments, the fragments have an average length from about 50 to about 2,000 nucleotides. In some embodiments, the fragments have an average length from about 100-2,500, 10-1,000, 10-800, 10-500, 50-500, 50-250, or 50-150 nucleotides. In some embodiments, the fragments have an average length less than 500 nucleotides, such as less than 400 nucleotides, less than 300 nucleotides, less than 200 nucleotides, or less than 150 nucleotides. In some embodiments, the fragmentation is accomplished mechanically comprising subjection sample polynucleotides to acoustic sonication. In some embodiments, the fragmentation comprises treating the sample polynucleotides with one or more enzymes under conditions suitable for the one or more enzymes to generate double-stranded nucleic acid breaks. Examples of enzymes useful in the generation of polynucleotide fragments include sequence specific and non-sequence specific nucleases. Non-limiting examples of nucleases include DNase I, Fragmentase, restriction endonucleases, variants thereof, and combinations thereof. For example, digestion with DNase I can induce random double-stranded breaks in DNA in the absence of Mg⁺⁺ and in the presence of Mn⁺⁺. In some embodiments, fragmentation comprises treating the sample polynucleotides with one or more restriction endonucleases. Fragmentation can produce fragments having 5′ overhangs, 3′ overhangs, blunt ends, or a combination thereof. In some embodiments, such as when fragmentation comprises the use of one or more restriction endonucleases, cleavage of sample polynucleotides leaves overhangs having a predictable sequence. In some embodiments, the method includes the step of size selecting the fragments via standard methods such as column purification or isolation from an agarose gel.

In some embodiments, the 5′ and/or 3′ end nucleotide sequences of fragmented DNA are not modified prior to ligation with one or more adapter oligonucleotides. For example, fragmentation by a restriction endonuclease can be used to leave a predictable overhang, followed by ligation with one or more adapter oligonucleotides comprising an overhang complementary to the predictable overhang on a DNA fragment. In another example, cleavage by an enzyme that leaves a predictable blunt end can be followed by ligation of blunt-ended DNA fragments to adapter oligonucleotides comprising a blunt end. In some embodiments, the fragmented DNA molecules are blunt-end polished (or “end repaired”) to produce DNA fragments having blunt ends, prior to being joined to adapters. The blunt-end polishing step may be accomplished by incubation with a suitable enzyme, such as a DNA polymerase that has both 3′ to 5′ exonuclease activity and 5′ to 3′ polymerase activity, for example T4 polymerase. In some embodiments, end repair is followed by an addition of 1, 2, 3, 4, 5, 6, 7, 8, 9, 10, 11, 12, 13, 14, 15, 16, 17, 18, 19, 20 or more nucleotides, such as one or more adenine, one or more thymine, one or more guanine, or one or more cytosine, to produce an overhang. DNA fragments having an overhang can be joined to one or more adapter oligonucleotides having a complementary overhang, such as in a ligation reaction. For example, a single adenine can be added to the 3′ ends of end repaired DNA fragments using a template independent polymerase, followed by ligation to one or more adapters each having a thymine at a 3′ end. In some embodiments, adapter oligonucleotides can be joined to blunt end double-stranded DNA fragment molecules which have been modified by extension of the 3′ end with one or more nucleotides followed by 5′ phosphorylation. In some cases, extension of the 3′ end may be performed with a polymerase such as for example Klenow polymerase or any of the suitable polymerases provided herein, or by use of a terminal deoxynucleotide transferase, in the presence of one or more dNTPs in a suitable buffer containing magnesium. In some embodiments, target polynucleotides having blunt ends are joined to one or more adapters comprising a blunt end. Phosphorylation of 5′ ends of DNA fragment molecules may be performed for example with T4 polynucleotide kinase in a suitable buffer containing ATP and magnesium. The fragmented DNA molecules may optionally be treated to dephosphorylate 5′ ends or 3′ ends, for example, by using enzymes known in the art, such as phosphatases.

In some embodiments, each of the plurality of independent samples comprises at least about 1 pg, 10 pg, 100 pg, 1 ng, 10 ng, 20 ng, 30 ng, 40 ng, 50 ng, 75 ng, 100 ng, 150 ng, 200 ng, 250 ng, 300 ng, 400 ng, 500 ng, 1 μg, 1.5 μg, 2 μg, or more of nucleic acid material. In some embodiments, each of the plurality of independent samples comprises less than about 1 pg, 10 pg, 100 pg, 1 ng, 10 ng, 20 ng, 30 ng, 40 ng, 50 ng, 75 ng, 100 ng, 150 ng, 200 ng, 250 ng, 300 ng, 400 ng, 500 ng, 1 μg, 1.5 μg, 2 μg, or more of nucleic acid.

In another aspect, the invention provides compositions that can be used in the above described methods. Compositions of the invention can comprise any one or more of the elements described herein. In one embodiment, the composition comprises a plurality of target polynucleotides, each target polynucleotide comprising one or more barcode sequences selected from a plurality of barcode sequences, wherein said target polynucleotides are from two or more different samples, and further wherein the sample from which each of said polynucleotides is derived can be identified in a combined sequencing reaction with an accuracy of at least 95% based on a single barcode contained in the sequence of said target polynucleotide. In some embodiments, the composition comprises a plurality of first adapter oligonucleotides, wherein each of said first adapter oligonucleotides comprises at least one of a plurality of barcode sequences, wherein each barcode sequence of the plurality of barcode sequences differs from every other barcode sequence in said plurality of barcode sequences at least three nucleotide positions.

In one aspect, the invention provides kits containing any one or more of the elements disclosed in the above methods and compositions. In some embodiments, a kit comprises a composition of the invention, in one or more containers. In some embodiments, the invention provides kits comprising adapters, primers, and/or other oligonucleotides described herein. In some embodiments, the kit further comprises one or more of: (a) a DNA ligase, (b) a DNA-dependent DNA polymerase, (c) an RNA-dependent DNA polymerase, (d) random primers, (e) primers comprising at least 4 thymidines at the 3′ end, (f) a DNA endonuclease, (g) a DNA-dependent DNA polymerase having 3′ to 5′ exonuclease activity, (h) a plurality of primers, each primer having one of a plurality of selected sequences, (i) a DNA kinase, (j) a DNA exonuclease, (k) magnetic beads, (l) an enzyme comprising RNase H activity, (m) an RNA ligase, and (n) one or more buffers suitable for one or more of the elements contained in said kit. The adapters, primers, other oligonucleotides, and reagents can be, without limitation, any of those described above. Elements of the kit can further be provided, without limitation, in any of the amounts and/or combinations (such as in the same kit or same container) described above. The kits may further comprise additional agents, such as those described above, for use according to the methods of the invention. The kit elements can be provided in any suitable container, including but not limited to test tubes, vials, flasks, bottles, ampules, syringes, or the like. The agents can be provided in a form that may be directly used in the methods of the invention, or in a form that requires preparation prior to use, such as in the reconstitution of lyophilized agents. Agents may be provided in aliquots for single-use or as stocks from which multiple uses, such as in a number of reaction, may be obtained.

In one embodiment, the kit comprises a plurality of first adapter oligonucleotides, wherein each of said first adapter oligonucleotides comprises at least one of a plurality of barcode sequences, wherein each barcode sequence of the plurality of barcode sequences differs from every other barcode sequence in said plurality of barcode sequences at least three nucleotide positions, and instructions for using the same. First adapters comprising different barcode sequences can be supplied individually or in combination with one or more additional first adapter having a different barcode sequence. In some embodiments, the kit further comprises a plurality of second adapter oligonucleotides. Second adapter oligonucleotides can be supplied separately from or in combination with one or more first adapters, and/or one or more different second adapters. Combinations of first and second adapters can be supplied in accordance with combinations described above.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

### Fragmentation and Repair of Sample Nucleic Acid

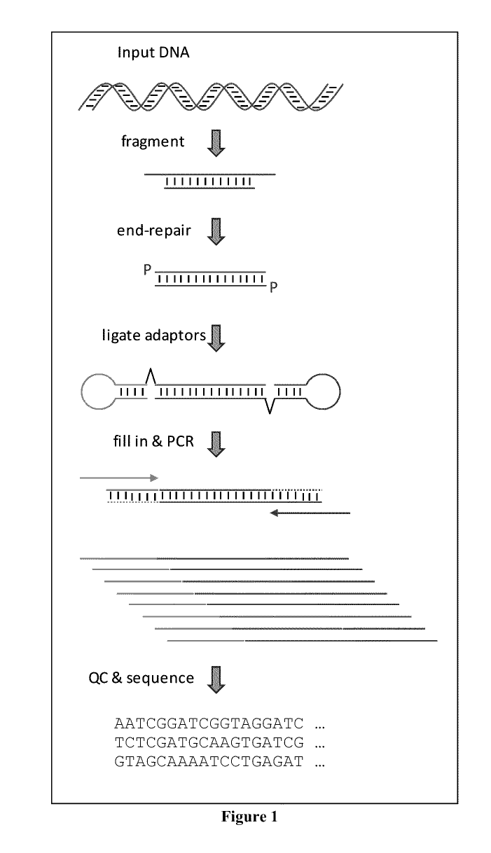

The sample comprising target polynucleotides (“sample”) used in this example is human genomic DNA. In order to fragment the nucleic acid, 1 ug to 5 ug are diluted in 120 uL of TE, and the dilution is subjected to mechanic fragmentation using a Covaris S-series sonication instrument (Covaris, Inc.) with the following setting: duty cycle=10, intensity=5, cycles/burst=100, time=10 minutes, and sample volume=120 uL. Fragmented nucleic acid is the purified with SPRI beads (Beckman Coulter, Inc.) at a ratio of 1:1.8 (sample:beads). DNA is eluted from the beads with 40 uL of TE, and quantified, such as by using a Nanodrop, Quibit, or similar DNA quantitation instrument, or spectrophotometrically. Fragmentation products having 5′ overhangs, 3′ overhangs, non-phosphorylated 3′ ends, and/or phosphorylated 3′ ends are then end-repaired using a blend of enzymes that specifically eliminate overhangs and restore end residues to the appropriate 5′ phosphorylation and 3′ hydroxyl configuration. For end-repair using the Quick Blunting kit (New England Biolabs, Inc.), 100-200 ng of fragmented DNA is combined with 1.25 uL 10× quick blunt buffer, 1.25 uL 1 mM dNTP mix, and water to a final volume of 12 uL. This combination is mixed well, spun down in the tube, and 0.5 uL of quick blunt enzyme (a combination of T4 DNA polymerase and T4 polynucleotide kinase) is added, followed by incubation at room temperature for 30 minutes, and inactivation at 70° C. for 10 minutes. Nucleic acid prepared according to the methods of this example can be stored at −20° C. or used immediately is subsequent ligation reaction to join target polynucleotide fragments to adapters. A diagram of the steps in the procedure, including fragmentation, end-repair, adapter ligation, adapter fill-in, amplification, and sequencing is shown in FIG. 1.

### Example 2

### Effects of Target Polynucleotide to Adapter Ratios on Library Construction

The present example examines the effects of varying target polynucleotide to adapter ratios on the construction of a collection of adapter tagged target polynucleotides (or “library”). The sample comprising target polynucleotides (“sample”) used in this example is prepared as described in Example 1. The first adapter in this example consists of SEQ ID NO: 7. The second adapter consists of SEQ ID NO: 8. One of the primers used in the amplification step of this example consists of SEQ ID NO: 9, and the other primer in the pair consists of SEQ ID NO: 10. Ligation reactions are prepared such that each contains 10 uL of 2× ligation buffer, 4 uL of sample nucleic acid, 4 uL of combined adapters, 1 uL of water (5 uL in reaction lacking sample or adapters), and 1 uL ligase. In addition to buffer, water, and ligase, the tested reactions consist of: no sample (reactions 1 to 4), 20 ng of sample (reaction 5 to 8), and 200 ng of sample (reactions 9 to 12) in combination with (in reaction order) 1 uM adapters, 0.2 uM adapters, 0.04 uM adapters, or 0.008 uM adapters. Additional controls, in addition to buffer, water, and ligase, consist of the following, listed by reaction number: (13) 200 ng of sample without adapters, (14) 200 ng of sample and 1 uM first adapter only, (15) 200 ng of sample and 1 uM second adapter only, (16) water only), (17) 1 uM first adapter only, and (18) 1 uM second adapter only. Ligation reactions are incubated at room temperature for 10 minutes. Ligation products are then subjected to an amplification step, with each amplification reaction containing 3 uL water, 2 uL 5×PCR buffer, 1 uL 25 mM MgCl₂, 1 uL 10 uM first primer, 1 uL 10 uM second primer, 0.5 uL 10 mM dNTPs, 0.5 uM DMSO, 0.1 uL Expand enzyme mix, 0.1 uL Taq polymerase, and 1 uL of one of the ligation reactions. Amplification reaction mixtures were then subjected to the following thermal cycling sequence: 1 cycle of 72° C. for 2 minutes, 95° C. for 2 minutes; 10 cycles of 95° C. for 30 seconds, 60° C. for 30 seconds, 72° C. for 1 minute; 20 cycles of 95° C. for 30 seconds, 60° C. for 30 seconds, 72° C. for 70° C. seconds; 72° C. for 7 minutes; hold at 10° C. until proceeding. The first cycle in this process serves to extend the 3′ ends of target polynucleotides using the adapters ligated to the 5′ end as template (the “fill in” reaction), yielding a double-stranded DNA adapter tag. At the end of thermal cycling, 2 uL of 6× loading dye is added to each reaction, and 5 uL of the resulting mixture is loaded on a 2% agarose gel in TAE. Gels are imaged to illuminate the DNA products resulting from ligation and amplification.

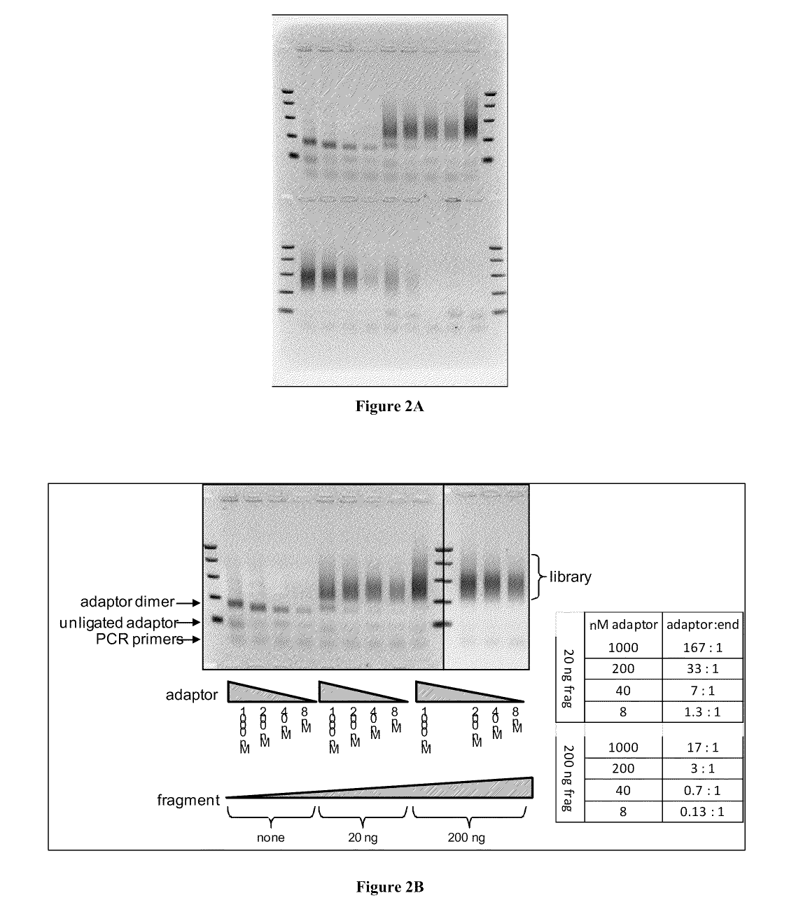

A sample result is shown in FIG. 2A. The top half of FIG. 2A contains, in lanes from left to right, ladder, reactions 1 to 9, and ladder. The lower half of FIG. 2A contains, in lanes from left to right, ladder, reactions 10 to 18, and ladder. Lanes 1 to 4 and 13 to 18 show that both sample nucleic acid and both adapters are required for efficient amplification of target polynucleotides. FIG. 2B provides a side-by-side comparison of reactions 1 to 12, in order from left to right, in addition to lanes containing ladder. The results indicate that, under these conditions, amplified libraries can be obtained using a first and second hairpin adapter, that the higher sample amount decreases the formation of primer dimers, and amplification yield remains relatively constant with decreasing adapter input.

### Example 3

### Barcoded Adapters and Sample Source Identification

Nucleic acid is isolated from samples derived from 16 individuals using standard methods. Isolated polynucleotide samples are processed independently as in Example 1. Adapters are then ligated to target polynucleotides as in Example 2, with each sample being joined to a first adapter having a different barcode and a second adapter consisting of SEQ ID NO: 8. The first adapters are independently assigned to each of the samples, and have sequences provided by SEQ ID NOs: 11-26.

Target polynucleotides having 5′ overhangs comprising the adapter sequences are then filled in by 3′ end extension using the adapter sequences as template as in Example 2. Target polynucleotides are then also PCR amplified as in Example 2, using a pair of primers, one comprising SEQ ID NO: 84 and the other comprising SEQ ID NO: 85. The amplification products are then pooled and submitted for sequencing according to Illumina's Solexa sequencing platform (see e.g. www.illumina.com). Pooled sequencing data is then parsed on the basis of the barcodes contained in the sequencing reads to create 16 bins of sequencing data. The bins are then assembled as though each had been run independently, providing sorted and aligned sequencing data for 16 independent samples from a single pooled sequencing reaction.

### Example 4

### Use of Hairpin Adapters Comprising Heteroduplexes

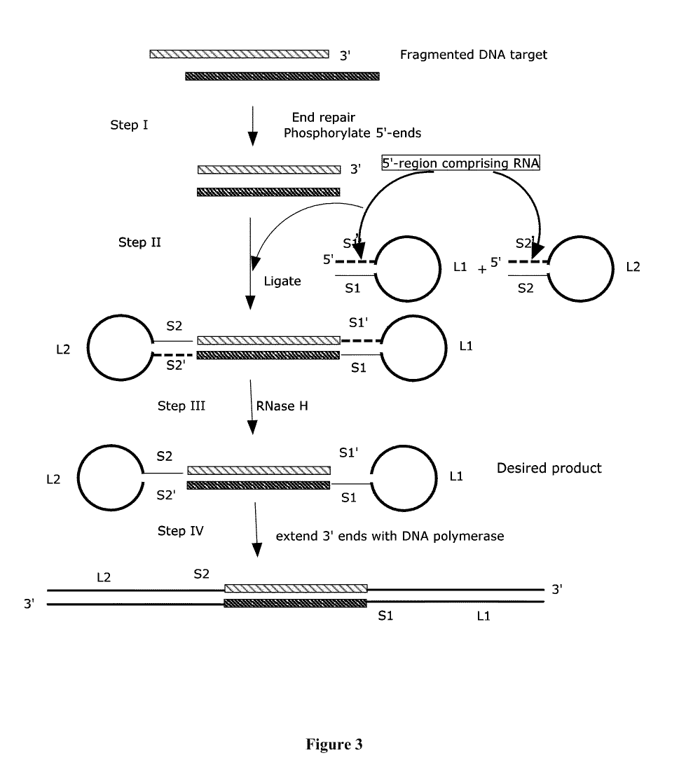

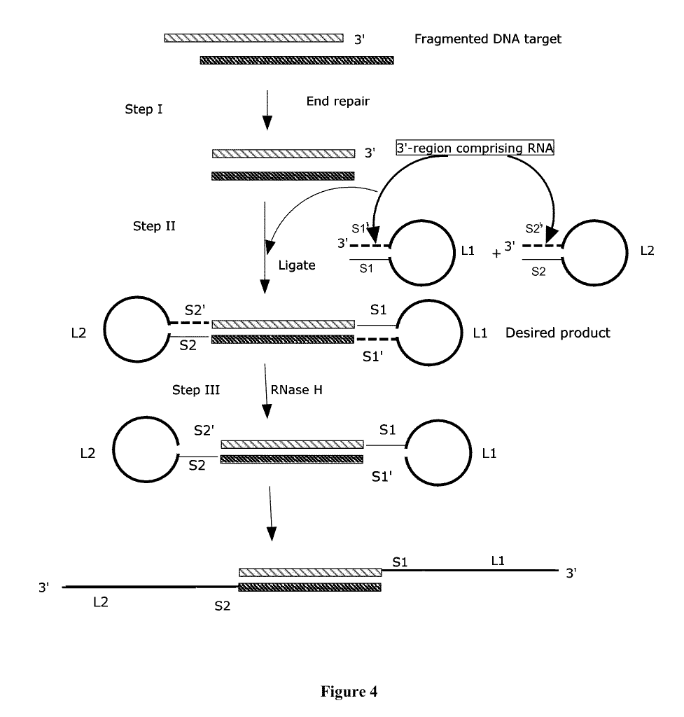

The sample comprising target polynucleotides (“sample”) used in this example is prepared as described in Example 1. First and second hairpin adapter oligonucleotides having stems involving both ends, forming blunt-ended structures, are ligated to target polynucleotides as in Example 2. With only target polynucleotides having 5′ phosphates, only the 3′ ends of the adapters are joined to the targets. As illustrated in FIG. 3, the hybridizable region of the 5′ ends of the adapters comprise RNA, while the sequence to which the 5′ ends hybridize comprises DNA. After ligation, RNase H cleaves the RNA of the RNA-DNA heteroduplexes, removing the secondary structure from the ligated adapters. DNA polymerase then extends the 3′ ends of the target polynucleotides using the remaining sequence of the ligated adapters as template, a step that does not require any strand displacement. This step is performed as in Example 2, and can also be followed by an amplification step using primers that hybridize to sequences derived from the adapters. The resulting adapter-tagged oligonucleotides are then sequenced using sequencing primers that hybridize to sequences derived from the adapters. In both FIG. 3 and FIG. 4, S1 (half of stem 1) is hybridizable to S1′ (the other half of stem 1), S2 (half of stem 2) is hybridizable to S2′ (the other half of stem 2), L1 is the loop sequence of the first adapter oligonucleotide, and L2 is the loop sequence of the second adapter oligonucleotide. Similarly, in FIG. 5, S1 is hybridizable to S1′, and L1 is the loop sequence of the adapter oligonucleotide. For the purpose of these illustrations, the sequences S1, S1′, S2, and S2′ correspond to sequences A, A′, B, and B′, respectively, as discussed above.

### Example 5

### Evaluation Ligation Efficiency of Various Hairpin Adapter Designs

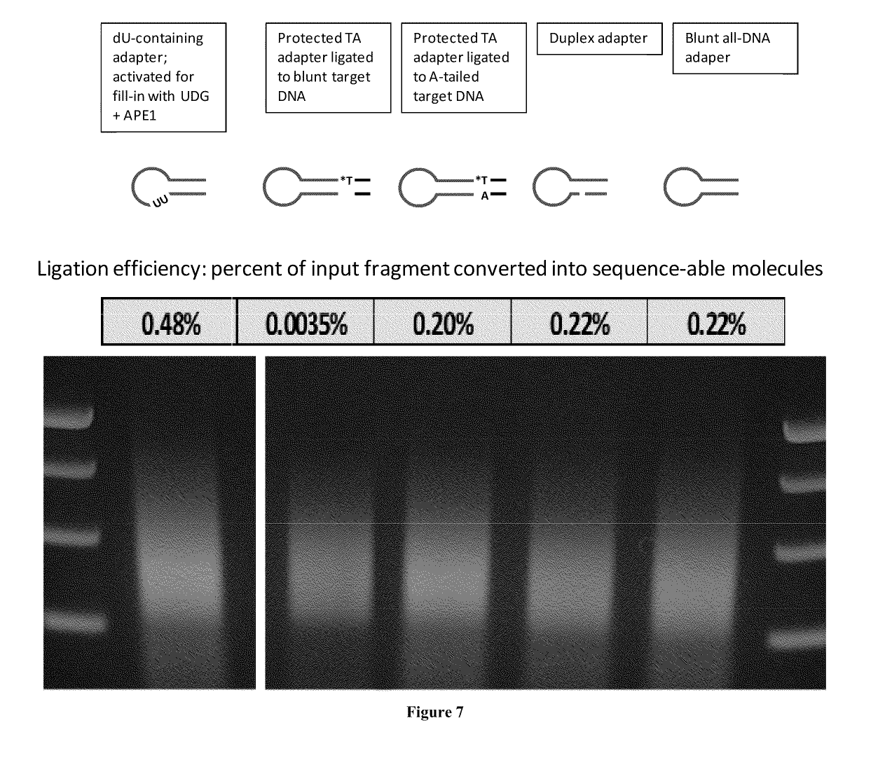

In this example, hairpin adapter oligonucleotides having different nucleotide compositions were evaluated for efficiency of ligation to target polynucleotides. Each ligation reaction included target polynucleotides and a pair of adapters, with each member of the pair having a different sequence but sharing a specified characteristic. As illustrated in FIG. 7, the various designs are, from left to right, blunt dU adapters, thymine-overhang adapters (ligated to blunt-ended target polynucleotides), thymine-overhang adapters (ligated to end repaired target polynucleotides modified to have 3′ adenine single-base overhangs), duplex hairpin adapters, and blunt all-DNA adapters. Blunt dU adapters included a dinucleotide of deoxy uridine nucleotides at the 5′ extreme end of the adapter loop (e.g. SEQ ID NO: 27 and SEQ ID NO: 28). Treatment of ligated material with UDG+APE1 cleaves the U bases and opens the loop for a subsequent fill in reaction (the remaining stem dissociates at the 72° C. temperature used in the fill-in reaction. Thymine-overhang adapters included an all-DNA sequence with a single thymine nucleotide 3′ overhang (e.g. SEQ ID NO: 35 and SEQ ID NO 36). Duplex hairpin adapters included a first or second hairpin oligonucleotide having a stem and a 3′ overhang (e.g. SEQ ID NO: 37 and SEQ ID NO: 38) hybridized to a short nucleotide (e.g. SEQ ID NO: 39), which hybridization included the 5′ terminus of the short nucleotide and the 3′ terminus of the hairpin oligonucleotide to form a stem effectively having a single-stranded break. Blunt all-DNA adapters consisted of DNA, with internal hybridization forming a blunt ended hairpin (e.g. SEQ ID NO: 40 and SEQ ID NO: 41). Example adapter sequences are provided by SEQ ID NOs: 27-43.

Human genomic DNA was fragmented as in Example 1. For end repair of fragmented genomic DNA, 52 uL of 191 ng/uL fragmented human genomic DNA was combined with 20 uL 10× quick blunt buffer, 20 uL 10×dNTPs, and 100 uL water, which was mixed before further adding 8 uL quick blunt enzyme mix. The end-repair reaction was incubated for 30 minutes at room temperature, and for 20 minutes at 75° C. For ligation to thymine-overhang adapters, 100 uL of end-repaired DNA was modified to have single adenine nucleotide 3′ overhangs (“tailed”) by adding 2 uL of 10 mM dATP (final concentration of 0.2 mM) and 8 uL of Klenow (3′->5′ exo minus), and incubating at 37° C. for 30 minutes followed by 75° C. for 20 minutes. Ligation reactions were prepared by combining 10 uL of 2× ligation buffer, 4 uL of end-repaired DNA or tailed DNA (approximately 200 ng total), 0.2 uL of each of a first and second adapter in a pair at concentrations of 10 uM, and 5 uL water, followed by mixing, adding 1 uL of T4 DNA ligase, and incubating at room temperature for 10 minutes. For ligation reactions using blunt dU adapters, 1 uL of a mixture of uracil DNA glycosylase (UDG) and apurinic endonuclease (APE) was added, followed by incubation at 37° C. for 10 minutes. After ligation, and cleavage where indicated, duplicate reactions were prepared for fill-in of 5′ overhangs by 3′ end extension for each adapter type ligation reaction. One of each duplicate fill-in reaction was further amplified by PCR using a pair of amplification primers (SEQ ID NO: 42 and SEQ ID NO: 43), while the other of each duplicate was used in the determination of ligation efficiency. Each fill-in/amplification reaction contained 8 uL of water, 2 uL of 10× amplification buffer, 2 uL of 25 mM MgCl₂, 2 uL of each amplification primer at 10 uM concentrations, 2 uL of one ligation reaction, 1 uL of DMSO, 1 uL of 10 mM dNTPs, and 0.2 uL of Taq polymerase enzyme. The fill-in/amplification reactions were incubated at 72° C. for 2 minutes. Amplification included 20 cycles of 94° C. for 30 seconds, 60° C. for 30 seconds, and 72° C. for 1 minute. Aliquots of amplification reactions were run on an agarose gel, the results of which are illustrated in FIG. 7.

Ligation efficiency was measured by quantitative PCR (qPCR). Ligation efficiency is defined as the percent of target molecules that are added to the library construction as input that end up in the final, amplified library. It is determined by using an existing library of known composition and concentration as a standard. Dilutions of this library are used to create a standard curve in a qPCR reaction. For measurement of unknowns, a calculated portion of the target input is removed following end-repair, ligation, and fill in. The qPCR signal from this sample is plotted on the standard curve to establish the quantity of properly ligated molecules. The difference between the measured signal and the known input establishes the ligation efficiency. qPCR reaction mixes included 12.5 uL of 2×SYBR mix (Clontech Laboratories, Inc.), 0.5 uL of each amplification primer at 10 uM concentrations, 5 uL of template (1/10 dilution of fill-in reaction, 1/100 dilution of fill-in reaction, library standard, or water for no template control), and 6.5 uL of water. Amplification of the qPCR reactions was conducted using standard methods, and ligation efficiency for each adapter design is provide below the illustration of the respective design in FIG. 7. In summary, for blunt dU adapters, thymine-overhang adapters (ligated to blunt-ended target polynucleotides), thymine-overhang adapters (ligated to end repaired target polynucleotides modified to have 3′ adenine single-base overhangs), duplex hairpin adapters, and blunt all-DNA adapters, efficiencies were about 0.48%, 0.0035%, 0.20%, 0.22%, and 0.22%, respectively. All adapter pairs produced comparable PCR amplification products.

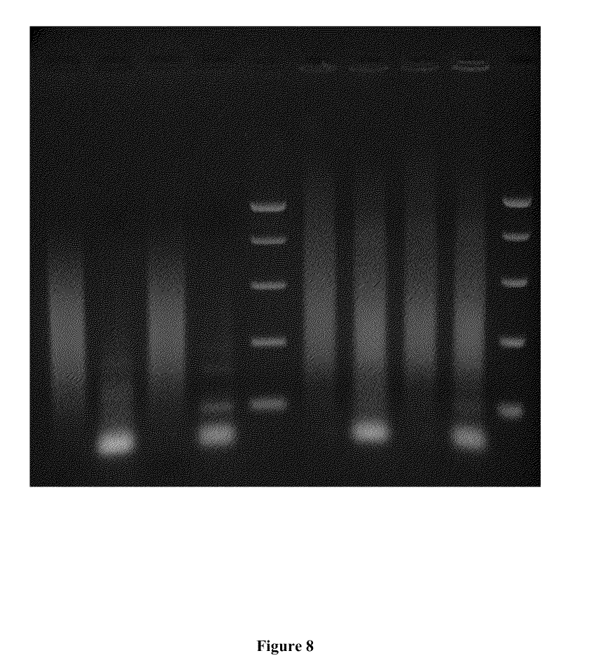

Examination of ligation products by agarose gel analysis indicated the presence of little or no adapter dimers. That amplification products contained target insert fragments of about the expected size was also confirmed. FIG. 8 shows a gel of samples of various reactions, with lane contents from left to right as follows: end repaired human genomic DNA, blunt all-DNA adapters, end repaired and A-tailed DNA, thymine overhang adapters, ladder, ligated end repaired DNA without adapters, end repaired DNA ligated with blunt all-DNA adapters, end repaired and A-tailed DNA ligated without adapters, end repaired and A-tailed DNA ligated with thymine overhang adapters, and ladder.

In some embodiments, a first duplex adapter in a pair of duplex adapters includes a first hairpin oligonucleotide having a stem and a 3′ overhang comprising a barcode hybridized to a short partner oligonucleotide comprising a sequence complementary to all or a portion of the 3′ overhang, including the barcode. The duplex adapter comprising two oligonucleotides may have a 5′ or 3′ overhang, or a blunt end when both oligonucleotides in the duplex are hybridized. The first duplex adapter may be paired with a second duplex adapter that is the same or different from the first duplex adapter, and the second duplex adapter may or may not contain a barcode. In general, the second duplex adapter may include a hairpin oligonucleotide having a stem and a 3′ overhang hybridized to a short nucleotide, such that the hybridized oligonucleotides together form an adapter having a 5′ or 3′ overhang, or a blunt end. Examples of first duplex adapters comprising a hairpin oligonucleotide having a barcode and paired with a short partner oligonucleotide includes the following sequence pairs: SEQ ID NO: 44 with SEQ ID NO: 45, SEQ ID NO: 46 with SEQ ID NO: 47, SEQ ID NO: 48 with SEQ ID NO: 49, SEQ ID NO: 50 with SEQ ID NO: 51, SEQ ID NO: 52 with SEQ ID NO: 53, SEQ ID NO: 54 with SEQ ID NO: 55, SEQ ID NO: 56 with SEQ ID NO: 57, SEQ ID NO: 58 with SEQ ID NO: 59, SEQ ID NO: 60 with SEQ ID NO: 61, SEQ ID NO: 62 with SEQ ID NO: 63, SEQ ID NO: 64 with SEQ ID NO: 65, SEQ ID NO: 66 with SEQ ID NO: 67, SEQ ID NO: 68 with SEQ ID NO: 69, SEQ ID NO: 70 with SEQ ID NO: 71, SEQ ID NO: 72 with SEQ ID NO: 73, and SEQ ID NO: 74 with SEQ ID NO: 75. In these sequences, barcodes are represented by the four bases at the 3′ end of the hairpin oligonucleotide in each pair of oligonucleotides of the duplex adapter, and the complement of the barcode represented by the four bases at the 5′ end of the short partner oligonucleotide in each pair of oligonucleotides of the duplex adapter. In general, each hairpin oligonucleotide of a pair is mixed with the corresponding short partner oligonucleotide in a ratio of 1:1.

### Example 6

### Evaluation Ligation Efficiency of Hairpin Adapters Comprising RNA

In this example, hairpin adapter oligonucleotides having different nucleotide compositions were evaluated for efficiency of ligation to target polynucleotides, as in Example 5. Each ligation reaction included target polynucleotides and a pair of adapters, with each member of the pair having a different sequence but sharing a specified characteristic. The adapter pairs included blunt all-DNA adapters, and blunt RNA with DNA:DNA end adapters. Blunt all-DNA adapters consisted of DNA, with internal hybridization forming a blunt ended hairpin (SEQ ID NO: 76 and SEQ ID NO: 77). Blunt RNA with DNA:DNA end adapters included a stem having one strand with 10 RNA bases at the 5′ end with five 5′ terminal DNA bases, hybridized to a second strand of all DNA (SEQ ID NO: 80 and SEQ ID NO: 81). Amplification of ligation reactions using these adapters was performed using a pair of amplification primers (SEQ ID NO: 82 and SEQ ID NO: 83). Example adapter and amplification primer sequences are provided by SEQ ID NOs: 76-83.

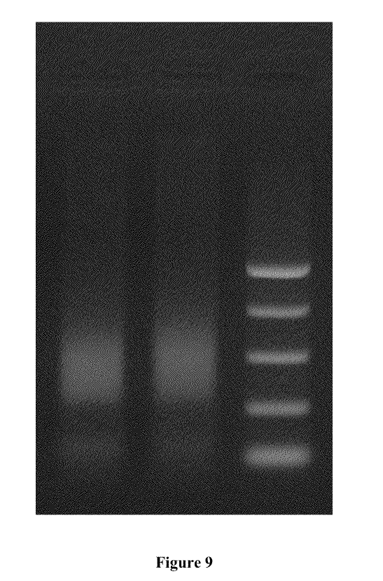

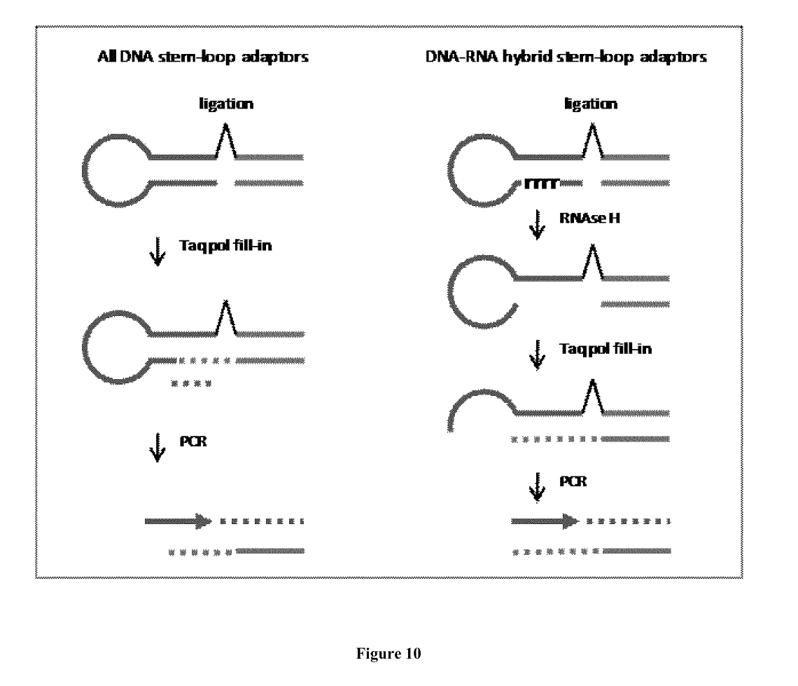

Fragmented target polynucleotides were prepared as in Example 5. Fragmented DNA was end-repaired as in Example 1 by combining, for each reaction, 4.2 uL of 47.5 ng/uL fragmented genomic DNA, 1.25 uL of 10× quick blunt buffer, 1.25 uL of 1 mM dNTP, 5.3 uL water, mixing, and adding 0.5 uL quick blunt enzyme. End-repair reactions were then incubated at room temperature (e.g. 20° C.-27° C.) for 30 minutes, and then incubated at 70° C. for 10 minutes. Ligation reactions were prepared in duplicate, using a full 12.5 uL end-repair reaction, combined with 12.5 uL of 2× quick ligase buffer, 0.25 uL of each adaptor in a pair at concentrations of 10 uM, and 1.25 uL of quick ligase. Ligation reactions were incubated at room temperature for 10 minutes prior to amplification. One ligation reaction in each duplicate was treated with RNase H in the amplification reaction mixture before beginning the amplification process. Both RNase H treated and untreated reactions were then subjected to 5′ overhang fill-in and ligation product amplification. Samples not treated with RNase H included 59 uL of water, 10 uL of 10×PCR buffer, 3 uL of 50 mM MgCl₂, 5 uL of each amplification primer at concentrations of 10 uM, 5 uL of DMSO, 2 uL of 10 mM dNTPs, 1 uL of Taq polymerase, and 10 uL of ligated template. Samples receiving RNase H treatment included 58 uL of water, 10 uL of 10×PCR buffer, 3 uL of 50 mM MgCl₂, 5 uL of each amplification primer at concentrations of 10 uM, 5 uL of DMSO, 2 uL of 10 mM dNTPs, 1 uL of Taq polymerase, 1 ul of RNase H, and 10 uL of ligated template. For samples receiving RNase H treatment, thermal cycling for amplification was preceded by incubation at 37° C. for 10 minutes (non-amplified, RNase H treated samples used as a baseline for quantification included additional steps of 72° C. for 2 minutes, and a 10° C. hold step). Amplification reaction mixtures were then subjected to the following thermal cycling sequence for fill-in and amplification: 1 cycle of 72° C. for 2 minutes; 20 cycles of 94° C. for 45 seconds, 55° C. for 30 seconds, and 72° C. for 90 seconds; 1 cycle of 72° C. for 7 minutes; and rest at 10° C. A 2% agarose gel containing 8 uL samples of PCR amplification reactions is shown in FIG. 9, with lanes from left to right corresponding to blunt RNA with DNA:DNA end adapter ligation products, blunt all-DNA adapter ligation products, and DNA ladder. A schematic diagram of the ligation between the 3′ end of the adapter and 5′ end of the target, RNase H treatment (where applicable), fill-in, and amplification reactions at one end of a target DNA is provided in FIG. 10.

Ligation efficiency was measured for each pair of adapters with or without RNase H treatment as described in Example 5. Each qPCR reaction in this example comprised 5 uL of 2×SYBR Green Mix, 0.4 uL of each amplification primer, 2.2 uL water, and 2 uL of diluted ligation reaction, for a total of 10 uL for each qPCR reaction. Ligation efficiencies for blunt all-DNA adapters with RNase H treatment, blunt all-DNA adapters without RNase H treatment, blunt RNA with DNA:DNA end adapters with RNase H treatment, and blunt RNA with DNA:DNA end adapters without RNase H treatment were 0.20%, 0.37%, 0.28%, and 0.13%, respectively. Successfully ligated and amplified fragments may be used as a next-generation sequence library.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for multiplex sequencing comprising sequencing a plurality of target polynucleotides in a single reaction chamber, wherein said target polynucleotides are from two or more different samples; and identifying the sample from which each of said sequenced target polynucleotides is derived with an accuracy of at least 95% based on a single barcode contained in the sequence of said target polynucleotide.

2. The method of claim 1, wherein said target polynucleotides comprise one or more sequences with which the sequencing reaction is calibrated.

3. The method of claim 1, wherein each barcode differs from every other barcode at at least three nucleotide positions.

4. The method of claim 1, wherein said identification is accurate after the mutation or deletion of a nucleotide in said barcode.

5. A method of producing adapter-tagged target polynucleotides from a plurality of independent samples, the method comprising:
a) providing a plurality of first adapter oligonucleotides, wherein each of said first adapter oligonucleotides comprises at least one of a plurality of barcode sequences, wherein each barcode sequence of the plurality of barcode sequences differs from every other barcode sequence in said plurality of barcode sequences at least three nucleotide positions; and
b) joining at least one of said first adapter oligonucleotides to said target polynucleotides of each of said samples, such that no barcode sequence is joined to said target polynucleotides of more than one of said samples.

6. The method of claim 5, further comprising (c) joining at least one of a plurality of second adapter oligonucleotides to said target polynucleotides of each of said samples from step (b), such that at least some of said target polynucleotides comprise said first adapter oligonucleotide at one end and said second adapter oligonucleotide at the other end.

7. The method of claim 6, further comprising pooling the target polynucleotides from step (c).

8. The method of claim 7, further comprising sequencing one or more of said polynucleotides in said pool.

9. The method of claim 8, further comprising identifying the sample from which a target polynucleotide is derived based on a barcode sequence to which it is joined.

10. The method of claim 6, wherein one or more of said adapter oligonucleotides comprises at least one of SEQ ID NO: 1 and SEQ ID NO: 2.

11. (canceled)

12. The method of claim 6, wherein one or more of said adapter oligonucleotides comprise at least one of a hairpin structure and an oligonucleotide duplex.

13-15. (canceled)

16. The method of claim 1, wherein said target polynucleotides comprise fragmented sample polynucleotides.

17-22. (canceled)

23. The method of claim 16, wherein said fragments have an average length of 50-500 nucleotides.

24-38. (canceled)

39. The method of claim 1, wherein said plurality of barcode sequences includes sequences selected from the group consisting of: AAA, TTT, CCC, GGG, AAAA, CTGC, GCTG, TGCT, ACCC, CGTA, GAGT, TTAG, AGGG, CCAT, GTCA, TATC, ATTT, CACG, GGAC, TCGA, AAAAA, AACCC, AAGGG, AATTT, ACACG, ACCAT, ACGTA, ACTGC, AGAGT, AGCTG, AGGAC, AGTCA, ATATC, ATCGA, ATGCT, ATTAG, CAACT, CACAG, CAGTC, CATGA, CCAAC, CCCCA, CCGGT, CCTTG, CGATA, CGCGC, CGGCG, CGTAT, CTAGG, CTCTT, CTGAA, CTTCC, GAAGC, GACTA, GAGAT, GATCG, GCATT, GCCGG, GCGCC, GCTAA, GGAAG, GGCCT, GGGGA, GGTTC, GTACA, GTCAC, GTGTG, GTTTT, TAATG, TACGT, TAGCA, TATAC, TCAGA, TCCTC, TCGAG, TCTCT, TGACC, TGCAA, TGGTT, TGTGG, TTAAT, TTCCG, TTGGC, and TTTTA.

40-62. (canceled)

63. A kit useful in the generation of adapter-tagged target polynucleotides, the kit comprising a plurality of first adapter oligonucleotides, wherein each of said first adapter oligonucleotides comprises at least one of a plurality of barcode sequences, wherein each barcode sequence of the plurality of barcode sequences differs from every other barcode sequence in said plurality of barcode sequences at least three nucleotide positions, and instructions for using the same.

64-82. (canceled)

83. A method of producing adapter-tagged target polynucleotides, the method comprising:
a) providing a plurality of first adapter oligonucleotides, wherein each of said first adapter oligonucleotides comprises a 5′ end comprising sequence A and a 3′ end comprising sequence A′, and further wherein A is hybridizable to A′, one of A or A′ comprises DNA, and the other of A or A′ comprises RNA and 5 or more terminal DNA nucleotides; and,
b) joining at least one of said first adapter oligonucleotides to at least one of said target polynucleotides.

84. The method of claim 83, further comprising the step of cleaving RNA with an enzyme that cleaves RNA from an RNA-DNA heteroduplex.

85. The method of claim 84, further comprising performing the step of extending one or more 3′ ends of said target polynucleotides, using said one or more joined adapter oligonucleotides as template.

86. The method of claim 83, further comprising joining at least one of a plurality of second adapter oligonucleotides to said target polynucleotides of each of said samples from step (b), such that at least one of said target polynucleotides comprises said first adapter oligonucleotide at one end and said second adapter oligonucleotide at the other end.

87. The method of claim 86, wherein each of said second adapter oligonucleotides comprises a 5′ end comprising sequence B and a 3′ end comprising sequence B′, and further wherein B is hybridizable to B′, one of B or B′ comprises DNA, and the other of B or B′ comprises RNA and 5 or more terminal DNA nucleotides.

88. The method of claim 83, wherein each of said first adapter oligonucleotides comprises a barcode sequence.

89-92. (canceled)
